(19) United States
(12) Patent Application Publication
(10) Pub. No.: US 2025/0132036 A1
(43) Pub. Date: Apr. 24, 2025

(54) METHOD OF PERFORMING A CLINICAL ASSESSMENT

(71) Applicant: Novoic Ltd., London (GB)

(72) Inventors: Jack Weston, London (GB); Emil Fristed, London (GB)

(21) Appl. No.: 18/493,205

(22) Filed: Oct. 24, 2023

(51) Int. Cl. G16H 50/20

(52) U.S. Cl. CPC G16H 50/20

(57) ABSTRACT

A computer-implemented method (200) is disclosed for performing a clinical assessment, the method comprising: providing a first input (206) to a machine learning model (208), the first input comprising template data encoding a template for carrying out a part of the clinical assessment; providing a second input (210) to the machine learning model, the second input comprising assessment data recorded during the clinical assessment; wherein the first input is provided to the machine learning model to condition the machine learning model to provide an output (212) based on the second input for use in the clinical assessment; and using the output from the machine learning model to perform the clinical assessment.

### FIELD OF THE INVENTION

The invention relates to computer-implemented methods for performing clinical assessments. In particular, the invention relates to automated methods for administering and analysing data recorded during clinical assessments.

### BACKGROUND

Clinical assessments generally involve a dialogue between a clinical interviewer and a patient, either in person or remotely, and are intended to provide assessment of a health condition of the subject. Such clinical assessments may be applied in neuropsychological testing to assess a cognitive impairment, for example a neurodegenerative condition, a psychiatric condition, a mental health condition, a neuro-developmental condition or a brain injury. Such assessments generally involve a sequence of instructed tasks intended to test cognitive function, where the patient's responses are scored and the resulting scores used to provide data on which an assessment of the health condition can be based. Such clinical assessments follow a defined structure with the sequence of instructed tasks provided as a standard operating procedure (SOP) or a template for use by the administrator of the clinical assessment.

Some examples of clinical assessments include the Wechsler Logical Memory Test and Novoic's Automated Story Recall Task, wherein the interviewer presents a story and asks the patient to repeat it, both immediately and after a delay. The interviewer has specific prompting instructions based on the patient's behaviour, set out the SOP. Another example is the Mini-Mental State Exam (MMSE), which comprises a list of questions and tasks presented by the interviewer, including questions and drawing tasks. Thus, input from the patient in some assessments can be in the form of drawings as well as spoken replies. A further example is the Clinical Dementia Rating (CDR), which is a long, semi-structured interview.

Once administered, clinical assessments also need to be evaluated or scored, which is known as rating. Rated clinical assessments may need to be reviewed, for quality control purposes or to obtain a second medical opinion. Administering, evaluating and reviewing clinical assessments are time consuming tasks that are error-prone due to human fallibility and often lack consistency. Further, in many jurisdictions there is a shortage of qualified personnel who can administer such clinical assessments, meaning inadequate provision of services to diagnose and monitor neuropsychological conditions.

Although there have been some attempts to automate clinical assessments and the various constituent tasks, until now the results of these attempts have not been adequate. No existing method has successfully automated the complex sequence of stages required to fully implement such clinical assessments and there remain a number of technical challenges to doing so. For example, automating rating of a clinical assessment can be difficult without a high-quality transcription of the assessment and existing automated transcription methods generally do not provide sufficiently accurate results for use in rating or reviewing clinical assessments. On the other hand, human-based transcription is labour intensive and not suitable to perform at scale.

There are further complications that make performing automated rating and review of clinical assessments difficult. One such complication is that practical rating and review processes require not only high-quality transcriptions, but also diarised transcriptions that identifies the relevant speaker of each speech element. Manual diarisation is similarly labour intensive and prone to mistakes. Clinical assessments may also deviate from a prescribed structure, making it difficult to determine automatically the clinical relevance of a section of speech, even if the transcript is diarised. Rating, review, and administration of clinical assessments are thus multi-stage processes that have proven thus far to be prohibitively complex to perform effectively and reproducibly in an automated manner. There is thus a need to provide more effective review, rating and administration methods for clinical assessments with reduced human involvement.

It is an object of the present invention to make progress in addressing these issues.

### SUMMARY OF INVENTION

According to a first aspect of the present invention, there is provided a computer-implemented method for performing a clinical assessment, the method comprising: providing a first input to a machine learning model, the first input comprising template data encoding a template for carrying out a part of the clinical assessment; providing a second input to the machine learning model, the second input comprising assessment data recorded during the clinical assessment; wherein the first input is provided to the machine learning model to condition the machine learning model to provide an output based on the second input for use in the clinical assessment; and using the output from the machine learning model to perform the clinical assessment.

In this way, the machine learning model provides a higher quality output that is better tailored to the specific context of the clinical assessment. The template encoded in the first input provides additional information to the machine learning model about the type of content that can be expected in the assessment data in the second input. This allows the machine learning model to interpret the assessment data more accurately to produce a higher quality output. In particular, conditioning the model on the SOP biases the models output in view of the expected structure and content of the assessment. For instance, the first input provides information allowing the machine learning model to disambiguate a spoken or written word in the assessment data that could have one of several spellings or meanings. Importantly, conditioning machine learning models in this way has been found to produce high quality outputs even for general purpose models that have not been specifically trained to perform tasks for the clinical assessment. In particular, the method may use pre-trained generative models, with little or no task-specific training, with the models simply conditioned by encoding an inputting the SOP or “template” into the model. Thus, large amounts of time-consuming model training can be avoided.

This approach has been found to be effective for performing a wide range of tasks, based on input and output data of various contents and formats, that are directly or indirectly related to making a clinical assessment. Thus, a wide range of possible types of outputs can be generated using this technique. Accordingly, it should be understood that “performing a clinical assessment” is intended to encompass not only administering a clinical assessment, but also rating (or “scoring”) the assessment, reviewing the administration of or any other aspect of a clinical assessment, or analysing a clinical assessment in any step between administering the assessment and rating or reviewing it. Examples of such analysis tasks include preparing a transcript of the assessment for subsequent review, translating the assessment, performing speaker diarisation, or segmenting the assessment data into segments based on the SOP. Thus, the method may also be described as “performing, analysing, rating or reviewing a clinical assessment”. Similarly, the template that is “for carrying out a part of the clinical assessment” can be related to rating or reviewing the clinical assessment as well as administering the clinical assessment.

It would also be appreciated that the method could be applied when using a machine learning model to perform tasks related to any situation following a template or structured interview format, which may not be related to clinical assessments.

The skilled person would appreciate that the particulars of how the machine learning model is conditioned may depend on the particular type and structure of the chosen machine learning model. It would nevertheless be appreciated that in general the term “conditioning” refers to enabling a machine learning model to adjust internal representations in the model so that terms or objects relevant to the first input are more likely to appear in the output. The term conditioning may be defined as inputting an auxiliary input (i.e. the first input) encoding additional context into the model, such that the models output is modulated based on the additional contextual information. In the present invention the additional context is the template data, providing information on the structure or expected content of the clinical assessment, thus allowing the model to modulate its output in view of this. The model's internal representations are conditioned in the sense that each layer making up the model depends on the previous layer, where at least one earlier layer (such as an input layer) is based on the template encoded in the first input. Thus, the template information provided in the first input propagates through the model in a chain of dependency from the first input to the output. For an attention-based machine learning model (like a Transformer), these adjusted internal representations effectively reweight the importance of (a) positions along the input sequence, and (b) representation subspaces within a given input element representation. However, conditioning the internal representations in this way does not necessarily have this effect in all model types. Thus, the effect of conditioning could also be described as adjusting a distribution in the model or adjusting information processing in the model.

In some examples, such as for rating or review purposes, the template encoded in the first input provides instructions on how to complete a task, which the machine learning model will follow when producing the output. In other examples, the machine learning model can be tasked with preparing a transcription, in which case the template encoded in the first input provides context rather than literal instructions to follow. In either case, the output is conditioned to be more “relevant”, as mentioned above, to the template for carrying out the clinical assessment.

Preferably, the assessment data encodes the response of a subject during the clinical assessment, wherein the method comprises using the output to monitor or diagnose a health condition of the subject. In this way, the output can be used in the process of to diagnosing or monitoring a health condition. The response of the subject can include audio and/or text data corresponding to speech responses of the subject during an interview performed as part of the clinical assessment. In other examples, the response of the subject can also include data related to other types of tasks administered during the assessment, such as drawing- or movement-based tasks encoded by image or video data. The monitoring or diagnosis of the health condition need not be specific to a particular disease or condition or used as the sole basis for diagnosis or monitoring. The health condition can be a metric that evaluates one aspect of a subject's condition, which may be relevant to several specific diseases or diagnoses for use within a broader treatment or diagnosis process. For instance, the output could be used to monitor the subject's agitation levels, which can be relevant to both Alzheimer's disease as well as bipolar disorder.

The monitoring or diagnosis may be performed as part of: patient care, including questions about management and planning, treatment: identifying treatment needs, individualising treatment programs, and keeping abreast of patient's changing treatment requirements, treatment: evaluating treatment efficacy; research, both theoretical and applied; forensic questioning of the kind that are frequently performed by neuropsychologists.

Preferably, the health condition comprises one or more of: a neurodegenerative condition; a psychiatric condition; a mental health condition; a neuro-developmental condition; a traumatic brain injury. In this way, the health condition is related to the cognition of a subject, which can be monitored or diagnosed using clinical tests that are particularly suited for being analysed or administered using methods of the present invention, particularly by one or more tasks requiring a response that can be used to assess the presence and/or severity of such a condition.

Preferably, the machine learning model comprises a generative machine learning model. This type of model has been found to be most effective. More preferably, the generative machine learning model comprises a pre-trained deep learning sequence-to-sequence model. Deep learning models can be defined as multi-layer artificial neural networks.

In general, the machine learning model can be a deep learning or shallow model; preferably a deep learning model, which may have multiple layers and be trained with gradient-based methods; more preferably a sequence-to-sequence deep learning model (such as long short-term memory (LSTM) networks, or transformers). The machine learning model may preferably use fixed or learnt positional embeddings; more preferably a classic or variant of an attention method (encoder-decoder or decoder-only Transformers, e.g. T5/GPT); may be pre-trained on generic (i.e. not necessarily clinical) data; and utilise 0-shot, few-shot learning or learning by fine-tuning (i.e., learning performed after an initial pre-training stage).

Preferably, the pre-trained deep learning sequence-to-sequence model has been trained (or “fine-tuned”) by performing one or more of the following training tasks: an instruction following training task in which the machine learning model is trained to generate an output based on an instruction in an input; training based on input-output pairs using an output schema, in which the machine learning model is trained to generate an output structured according to the output schema; a reinforcement learning task with feedback provided by a human or another machine learning model. In this way, the machine learning model, which can be general purpose machine learning model such as a large language model, can be refined using these training tasks to produce more accurate or higher quality outputs. In this context, the training can also be referred to as “fine-tuning”, which can refer to task-specific training performed after initial general-purpose training.

Preferably, the machine learning model is trained to map an input data sequence to an output data sequence, where each of the input and output data sequence may comprise one or more of text data, audio data, image data, video data. In this way, the machine learning model can be configured to map various types (or combinations of types) of input data to various types (or combinations) of output data to perform a wide variety of tasks. In some specific examples, the machine learning model can be trained to map audio data to text data, which could be used to generate a transcription output; text data to audio data, which could be used to generate a speech output to administer a part of a clinical assessment; or audio and text data to audio and text data that has been mapped (“segmented”) to corresponding parts of the template encoded in the first input. Any other combination could be used in other examples.

Preferably, the clinical assessment comprises a task for assessing a cognitive function or a neurological health condition of a subject and the assessment data encodes the response of the subject during the task. Preferably, the assessment data also encodes responses or speech of the administrator of the task, and optionally any other participants in the clinical assessment. More preferably, the task comprises one or more of: a speech-based task, the assessment data encoding a spoken response of the subject in response to a prompt; a drawing task, the assessment data encoding a drawing provided by the subject in response to a prompt; an action task, the assessment data encoding an action performed by the subject in response to a prompt. Example tasks include questions in the Clinical Dementia Rating, and recall-based tasks, such as the Weschler Logical Memory Test, though it would be appreciated that the task could be any kind of clinical task performed by a subject during a clinical assessment.

The term “speech-based” task refers to any kind of verbal task as known in the art, answers to which could be represented in the assessment data using audio or text data. Thus, speech-based tasks can also be referred to as “Auditory-verbal assessments”. When the clinical assessment comprises a speech-based task, the machine learning model can map between either or both of audio and text data in the assessment data to either or both of text and audio in the output.

Preferably, the assessment data comprises one or more of audio data, text data, video data, image data.

Preferably, the template for administering the clinical assessment comprises one or more of: instructions for performing the clinical assessment; an output schema indicating how the output of the machine learning model should be formatted; example responses provided by a subject or administrator to tasks within the clinical assessment. In this way, the template provides context for the machine learning model that indicates the kind of responses or semantic content that can be expected in the assessment data so that the assessment data can be analysed more accurately. It is preferable for the template to include both sample administrator questions as well as sample subject or patient responses so that the machine learning model can be conditioned more completely. In the specific case of example responses by a subject of the clinical assessment, the example responses may include verbal disfluencies to condition the machine learning model to recognise and include verbal disfluencies in the output. It has been recognised that such disfluencies are clinically useful data. Consequently, conditioning the machine learning model to recognise, rather than ignore, a subject's disfluencies enables these to be captured in the output and thus taken into account in subsequent analysis or evaluation of the clinical assessment.

More preferably, the template comprises text data. In one example, the text data may be natural language text data that can be used to provide instructions for a human clinician to perform the clinical assessment.

Preferably, the first input and second input are combined and input into the machine learning model, such that the model is conditioned on the content of the template to provide an adapted output in which the probabilities of possible outputs are adjusted in view of the template. In this way, the “conditioning” provided using the template manifests in adjusted probabilities so that certain terms or features more “relevant” (as discussed above) to the clinical assessment are more likely to appear appropriately in the output. At the same time, features highly irrelevant to the clinical assessment are less likely to appear in the output.

Preferably, the method comprises: encoding the first input into a sequence of tokens representing the text content of the template; encoding the second input into a sequence of tokens representing the assessment data, where the assessment data comprises one or more of text, audio, video and image data; where the sequences of tokens are combined and input into the machine learning model. In this way, the assessment data can be transformed into a format that may be easier or more efficient for the machine learning model to process. For example where the input is text, the input may be encoded into a sequence of word-piece tokens, as is known in the art.

Preferably, the first input further comprises instructions on implementing the template data. The instructions can also be referred to as a “prompt” for the machine learning model. In this way, the machine learning model can be instructed to perform a particular task. In one example, the instructions can include a request to segment the input data into constituent questions and answers for a clinical assessment following the template encoded in the first input.

Preferably, the first input further comprises an output schema encoding how the output of the machine learning model must be structured. More preferably, the output schema comprises a structured text format, preferably comprising one of JSON, XML, HTML. In this way, the output is provided directly in a format that can be processed easily by other computer-based processes. For example, the output could be provided directly, without requiring a conversion from natural language, into a structured format that enables compatibility with existing web-based clinical systems. Any other structured text format could be used in other examples.

Preferably, the clinical assessment comprises a speech-based clinical assessment comprising tasks instructed by a human or computer-implemented administrator and spoken responses to the tasks provided by the subject (or “patient”); wherein: the template of the first input comprises text data defining intended content of the clinical assessment; the assessment data of the second input comprises speech data encoding a response of the subject to an instructed task, where speech data comprises one or both of text and audio data; wherein the machine learning model is a generative machine learning model trained to generate, based on the second input, an output usable to monitor or diagnose a health condition of the subject; wherein the method comprises conditioning the machine learning model on the first input to bias the machine learning model to adapt the generated output in view of knowledge of the intended content of the clinical assessment. This approach may be particularly suitable for health conditions related to cognition, which can often be assessed using speech-based tasks. The term “intended content” can include a sample or ideal structure of the clinical assessment, possible questions to be asked by the interviewer as well as sample responses of the subject. Therefore, “intended content” can also be referred to as “example content”, “expected content”, or “content”. In other preferable examples, the assessment data additionally comprises speech data encoding administrator speech.

Preferably, the output comprises a transformed version of the assessment data usable to monitor or diagnose a health condition. For example, the assessment data can be an audio recording of an interview performed as part of the clinical assessment and the output can be a text-based transcription. In a further example, the assessment data can comprise a text transcription, optionally coupled with a corresponding audio recording, and the output can be labelled and partitioned audio files and text transcripts. The labelling can “diarise” each file to identify the participant in an interview of the clinical assessment that utters each section of speech. Transforming the assessment data enables automated administration to be performed, for instance by enabling subsequent text-based analysis of an audio recording of the clinical assessment.

Preferably, the output comprises one or more of: a transcription of the input speech data; a diarised version of the input speech data, where sections of the speech data are attributed to different participants in the clinical assessment data; a segmented version of the input speech data, in which the input speech data is segmented according to a structure of the clinical assessment defined in the template. These outputs are useful for enabling subsequent analysis of a clinical assessment by a human clinician or computer-implemented rating or review system. Additionally, these outputs can be highly labour intensive for a human to generate, and can be prone to errors when performed by humans.

Preferably, the assessment data comprises audio data encoding speech recorded during the clinical assessment and the machine learning model comprises a transcription model, the transcription model comprising a generative audio-to-text model trained to output text data comprising a transcript of the speech, the method comprising: conditioning the transcription model by inputting template data encoding one or both of a script for the speech-based assessment and a sample subject response, thereby conditioning the model to assign a higher probability to words more likely to be produced during the task. More preferably, the template data includes a sample patient response including disfluencies to condition the transcription model to include disfluencies in the transcription. As discussed above, disfluencies have been found to be clinically relevant information that can be an indicator of certain health conditions. Conditioning the machine learning model to include disfluencies enables the machine learning model to produce an output transcript containing more clinically relevant information that can be lost or filtered out in existing machine learning model based transcription approaches.

Preferably, the machine learning model comprises a rating model, the rating model comprising a machine learning model for outputting a rating indicating the subject's performance in an assessment task based on the input assessment data, the method comprising: providing a first input to the rating model, the first input comprising template data encoding one or both of an administration template comprising an intended format of the clinical assessment and a rating template comprising instructions for rating a subject's response to an assessment task; providing a second input to the machine learning model, the second input comprising assessment data encoding the subject's response to an assessment task; wherein the first input is provided to the machine learning model to condition the rating model to provide a rating based on the assessment data in view of the template data; and receiving a rating of an assessment task. The term “rating” refers to “rating” as used in clinical contexts. In clinical settings, rating is performed to score or evaluate a particular clinical task, and can follow a standardised “rating sheet”. The rating model can be any type of machine learning model suitable for rating a subject's performance in accordance with the administration and/or rating template. Advantageously, the rating model is not necessarily trained for the specific task of rating a task performed by the subject. This is made possible because the conditioning provided by the first input is sufficient for a general purpose model to perform the rating task. In one example, the machine learning model can be a pre-trained transformer model. The assessment data preferably also comprises data encoding responses of the administrator of the clinical assessment, such as specific questions that were asked of the subject.

Preferably, the method further comprises outputting an indication of a health condition of the subject based on the rating of the assessment task. In this way, a positive or negative indication of a health condition can be determined automatically, saving time for in-demand human clinicians.

Preferably, the machine learning model comprises an administration model, the administration model comprising a machine learning model for automating the instruction of one or more tasks for monitoring or diagnosing a health condition of a subject, the method comprising: providing a first input to the administration model, the first input comprising template data encoding an administration template comprising instructions for administering a part of the clinical assessment; providing a second input to the machine learning model, the second input comprising assessment data recorded during the clinical assessment, the assessment data comprising data encoding a response of the subject to a task administered by the model; wherein the administration model maps the second input to a structured output usable to initiate an action to administer the clinical assessment; where the administration model is conditioned on the first input so that its outputs are determined in view of the administration template. In this way, a clinical assessment can be administered automatically, saving time for in-demand human clinicians and enabling the administration of clinical assessments in remote locations. As discussed above with respect to the rating model, advantageously the administration model can be any general-purpose machine learning model. The structured output can be based on previous outputs as well as responses of the subject. The structured output can be based on a previous number of speaker turns, for example the previous 10 responses of the subject and the previous 10 output responses of the administration model.

Preferably, the administration model is conditioned to provide an output usable to perform one or more of the following actions: wait for further response from the subject; prompt the user for more information in relation to a task; output a task or question from the administration template; respond to the subject as prescribed by an instruction in the template; calling an external API or a web-hook; updating a persistent state of the model, the persistent state encoding a current position in the clinical assessment. The persistent state can be maintained by an “agent” that tracks the ongoing state of the clinical assessment. The response as prescribed by an instruction in the template could include prompting for more info, moving onto another task/question, answering a query from a participant, alerting the user that the time is up for a timed question, initiate a subsequent stage of a task, or any other suitable response.

Preferably, wherein the administration model is trained to generate a structured text output encoding the action to call, the structured text preferably comprising a structured JSON format.

Preferably, the method further comprises: inputting the output of the administration model into a speech synthesis model, the speech synthesis model comprising a text-to-audio generative machine learning model trained to output synthesised speech based on a text input; such that the administration model outputs text encoding instructions to the subject based on the received response of the subject encoded in the assessment data, and the speech synthesis model generates an audio stream comprising instructions to the subject, thereby facilitating automated audio-verbal administration of the clinical assessment. In this way, clinical assessments can be administered in real-time in an audio format that may be more familiar and convenient for subjects or patients. Additionally, administering the clinical assessment using audio-verbal data, rather than text data, may be more feasible for certain subjects, such as for those with impairments that prevent reading of text-based instructions. The output text is also preferably based on the template encoded in the first input and previous responses of the administration model.

Preferably, the method further comprises: receiving a real-time stream of assessment data during the clinical assessment; and inputting sequential sections of the assessment data into the administration model in order to generate actions to administer the clinical assessment in real-time. The actions can include any suitable action, such as wait for the subject to finish speaking, administer the next question of the template, prompt the subject for further information, or initiate an external program or operation. This allows real-time autonomous administration of a clinical assessment.

Preferably, the stream of assessment data comprises audio data, and the method further comprises: inputting sequential sections of the audio data into a transcription model, the transcription model comprising a generative machine learning model trained to output text data comprising a transcript of an input section of audio data, wherein the transcription model is conditioned on the first input; the method further comprising inputting the text data output by the transcription model into the administration model, wherein the administration model is a text-to-text generative model trained to output structured text for initiating an action to administer the clinical assessment. In this way, the automated administration of the clinical assessment can be performed based on a high quality transcription that has been conditioned using the first input. As described above, the action be any suitable action, such as wait for the subject to finish speaking, administer the next question of the template, prompt the subject for further information, or initiate an external program or operation.

Preferably, the method further comprises inputting the assessment data into a rating model, the rating model comprising a machine learning model trained to output a rating indicating the subject's performance in an assessment task based on the input assessment data; wherein the rating model is conditioned by further inputting template data encoding a rating template comprising instructions for rating the assessment task. More preferably, the clinical assessment comprises a plurality of tasks for assessing a health condition of the subject, the method further comprising generating a rating for each task and based on the ratings outputting an assessment outcome relating to the health condition of the subject. In this way, an evaluation about a health condition can be made automatically.

Preferably, the clinical assessment is speech-based, which can also be referred to as based on an auditory-verbal assessment as discussed above.

Preferably, the assessment data comprises speech data encoding speech from an interview between an administrator and a subject, forming part of the clinical assessment.

Preferably, the second input comprises one or both of: a video recording of the clinical assessment and image data related to a drawing-based task of the clinical assessment. In this way, various tasks can be performed by the machine learning model in relation to non-speech-based clinical tasks, such as drawing-based recall tasks or physical tasks.

Preferably, the method further comprises providing a third input to the machine learning model, the third input comprising a first rating indicating a subject's performance in an assessment task, the first rating suitable for monitoring or diagnosing a health condition; wherein the template data includes instructions for reviewing the first rating, the second input includes assessment data including a subject response to a task of the clinical assessment, and the output comprises a review rating that evaluates the quality of the first rating. In this way, the machine learning model can be used to perform a quality review of the first rating. The task can be any kind of clinical task discussed previously, such as drawing or question-based tasks. Where the task relates to question-based tasks, the assessment data can comprise a transcript of the clinical assessment obtained previously using embodiments of the invention described above.

Similarly, the third input could comprise assessment data that is a complete record of the clinical assessment, including actions and speech of the administrator. Thus, the template data including instructions for reviewing the first rating can also include instructions for: (i) determining if the template was followed by the administrator (e.g., “did the administrator deliver the question(s) verbatim?/Was the deviation unacceptable?); (ii) determining if the clinical assessment was delivered properly with respect to various factors (“Did the administrator deliver the questions at an acceptable speech rate?”/“Did the administrator give the correct amount of time for the question?”/“Did the administrator ask an appropriate number of follow-up questions?”); (iii) reviewing a rating sheet (“did the administrator write down the correct items into the rating sheet?”); and/or (iv) rating accuracy (“are the administrator's ratings correct?”)

Preferably, the assessment data further comprises a completed rating sheet completed by an administrator and used in providing the first rating, where the template data comprises instructions for checking the rating sheet. In this way, the method can be used to quality-check ratings made by human raters and clinicians.

Preferably, the method further comprises: encoding each segment of the template data into a respective representation; splitting the assessment data into a plurality of sections and encoding each section into a respective representation; using a pairwise scoring algorithm to compute a similarity of each of the template segment representations with each of the assessment data representations; using an alignment algorithm to determine an optimal alignment of the plurality of sections of the assessment data with the segments of the template using the computed similarity between the template segment representations and the assessment data representations; using the optimal alignment to split the assessment data into segments corresponding to segments of the template; and providing one of the segments of the assessment data as an input to the machine learning model for analysing the assessment data.

In this way, the assessment data can be split into segments that are below a maximum context or input data size of many general-purpose machine learning models, particularly generative machine learning models. It has been found that using segments of the template still provides sufficient conditioning for the machine learning model to process the assessment data more accurately. Additionally, the use of smaller (and corresponding) segments of template and assessment data in the first and second inputs has been found to produce better outputs in some cases. Therefore, corresponding segments of any of the types of template and assessment data can be generated in this manner and used to perform any of the tasks described above, such as in transcribing or diarising an audio recording of a clinical interview.

Preferably, the assessment data comprises audio or text data encoding speech recorded during the clinical assessment in a first language and the machine learning model comprises a translation model, the translation model comprising a generative audio- and/or speech-to-text model for outputting text data comprising a translation of the speech into a second language, the method comprising: conditioning the translation model using the first input, thereby conditioning the model to assign a higher probability to words more likely to be produced during the clinical assessment.

In this way, speech recorded during a clinical interview can be translated more effectively, using the conditioning provided by the template in the first input. Translation in this manner can be performed as a pre-processing step before performing a different embodiment of the method described above, or may be performed iteratively in real-time when administering a clinical assessment where a participant's spoken language differs from a language of the administration model. Translation may also be performed to translate assessment data into a different language required for human or computer-implemented rating or review. As many general purpose machine learning models have often been trained on English language text corpora and perform best in English, the second language is preferably English. It would be appreciated that any two languages could be used in principle.

According to a further aspect of the present invention, there is provided a system for analysing a clinical assessment, the system comprising a processor configured to perform any of the embodiments of the method described above.

According to a further aspect of the present invention, there is provided non-transient computer readable medium comprising instructions which, when executed by a processor, cause the processor to perform the any of the embodiments of the method described above.

According to a second aspect of the present invention there is provided a computer-implemented method for segmenting assessment data, recorded during a clinical assessment, for analysis using a machine learning model, where the clinical assessment is performed according to a structured template comprising a plurality of segments, the method comprising: encoding each segment of the template into a respective representation; splitting the assessment data into a plurality of sections and encoding each section into a respective representation; using a scoring algorithm to compute a similarity of each of the template segment representations with each of the assessment data representations; using an alignment algorithm to determine an alignment of the plurality of sections of the assessment data with the segments of the template using the computed similarity between the template segment representations and the assessment data representations; using the optimal alignment to split the assessment data into segments corresponding to segments of the template.

In this way, the assessment data can be split into segments that are below a maximum context or input data size of many general-purpose machine learning models, particularly generative machine learning models. It has been found that using segments of the template still provides sufficient conditioning for the machine learning model to process the assessment data more accurately. Additionally, the use of smaller (and corresponding) segments of template and assessment data in the first and second inputs has been found to produce better outputs in some cases.

Preferably, the method further comprises providing one of the segments of the assessment data as an input to a machine learning model for analysing the assessment data.

It would be appreciated that assessment data split into segments using the second aspect of the invention could be used to split assessment data and provide the split assessment data as the second input in any of the embodiments of the first aspect of the invention described above.

### BRIEF DESCRIPTION OF DRAWINGS

Embodiments of the invention are now described, by way of example, with reference to the drawings, in which:

FIG. 1 is a schematic control diagram of a computer for performing embodiments of the invention;

FIG. 2 is flowchart for a method for analysing a clinical assessment according to an embodiment of the invention;

FIG. 3 is flowchart for a method for analysing a clinical assessment according to an embodiment of the invention;

FIG. 4 is flowchart for a method for analysing a clinical assessment according to an embodiment of the invention;

FIG. 5 is flowchart for a method for analysing a clinical assessment according to an embodiment of the invention;

FIG. 6 is flowchart for a method for analysing a clinical assessment according to an embodiment of the invention;

FIG. 7 is flowchart for a method for administering a clinical assessment according to an embodiment of the invention;

FIG. 8 is flowchart for a method for segmenting assessment data according to an embodiment of the invention; and

FIG. 9 is flowchart illustrating a series of steps of segmenting assessment data according to an embodiment of the invention.

### DETAILED DESCRIPTION

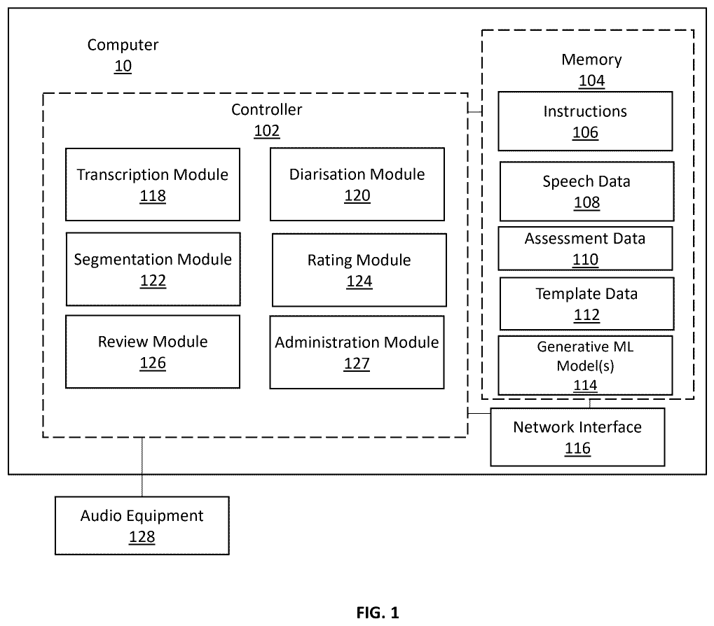

FIG. 1 is a schematic diagram of a computer suitable for carrying out methods of the invention.

The computer 10 comprises a controller 102 and a memory 104, the controller 102 including one or more processors for executing instructions 106 stored the memory 104. The instructions 106 can be executed by the controller 102 to carry out various modules implementing methods of the invention. The memory 104 can store speech data 108 from an interview of a clinical assessment in any suitable audio or text format, assessment data 110 relating to any other data (in addition to the speech data 108) recorded during a clinical assessment, template data 112 including instructions or guidance for administering, evaluating, or reviewing a clinical assessment, and at least one generative machine learning (ML) model 114. Alternatively, any of the items stored in memory 104 can be stored remotely in a distributed fashion across a network and accessed through a network interface 116.

The clinical assessment can be one or more tests designed to be evaluated to make or inform a diagnosis of a condition. The clinical assessment can also include more simple examples, such as a medical questionnaire, e.g., a series of questions about medical history and/or current health conditions, demographic information, medical history, inclusion criteria for clinical studies, clinical assessment questionnaires such as subjective memory decline, among other things. The assessment can be conducted in person or remotely via an audio or video call.

The speech data 108 can be any suitable format of text or audio file of speech between an interviewer and patient during an interview of a clinical assessment. Different modules of the controller 102, described further below, may require speech data in one of audio or text format, depending on particular purpose of the module.

The assessment data 110 can comprise any data recorded as part of the clinical assessment. For example, the assessment data 110 can include a video recording of the entire assessment, or video recordings related to a specific clinical test administered during the clinical assessment. In other examples, the assessment data 110 can include image files of a patient's response to a drawing-based task of the clinical assessment. The task data 110 may also be provided in any suitable format.

The template data 112 can include or be comprised solely of standard operating procedures (SOPs), which may be an official or non-official set of instructions for carrying out (“administering”), evaluating, or reviewing a particular clinical assessment. The SOPs may be in natural language. The template data 112 can include one or more scripts or guidance for carrying out a clinical interview, as part of an SOP or otherwise, in natural language. Each template may include a plurality of questions for the interviewer to ask the patient, optionally with corresponding example patient responses, where appropriate. Each template may have a hierarchical structure, such as assessment=>section=>question=>sample patient response, where each section may be directed to assessing a particular clinical criterion.

Templates in the template data 112 may be stored in any suitable structured format, such as a JSON, markdown, or XML format. In other cases, the templates may be an unstructured text or, less preferably, an image file, which can be encoded into a structured format by an encoder. Templates in the template data 112 can include schemas for worksheets to be filled in by a clinician or using the methods herein.

In the example methods below, the templates used are clinical SOPs, however any type of template for performing an aspect (e.g., administering, evaluating or reviewing) of a clinical assessment may be used alternatively or in addition.

The generative machine learning (ML) model 114 can be any suitable generative machine learning model type as known in the art. The model is “generative” as usually defined in the art, i.e., is configured to produce distributions over sequential data, such as text or audio, rather than distributions over classification labels. The generative machine learning model 114 may be a (multimodal) large language model that is general purpose or specialised to a specific task. The generative machine learning model 114 may be trained using reinforcement leaning by human feedback (RLHF) or otherwise.

It would be appreciated that different generative machine learning models may be used depending on the specific type of analysis performed in the methods described below.

The generative ML model 114 can take inputs of various formats to produce outputs of various other formats, for instance: text->text, text->audio, audio->text, {audio+text}->text, video->video, {video, text}->video} etc.

The example methods below utilise generative machine learning models, which have been found to be most effective. However, it is envisaged that the methods described herein could also be performed with non-generative machine learning models.

The controller 102 can execute instructions to operate various modules, such as a transcription module 118, a diarisation module 120, a segmentation module 122, a rating module 124, a review module 126, or an administration module 127. Operation and functionality of these modules is described further below with respect to various methods of analysing clinical assessments. The methods below can be performed offline to analyse completed assessments or interviews. Alternatively, as described with respect to the method 700, the methods can be performed live for an ongoing clinical interview.

The controller 102 may be in communication with and control various peripherals, such as audio equipment 128 for recording and/or generating audio signals. The audio equipment 128 may record or emit speech sounds to perform clinical interviews directly. Alternatively, such input and output audio signals can be transmitted through the network interface 116 to remote peripherals controlled by a different computer.

Some functionality of the various modules of the controller may be executed remotely over a network. For example, the computer 10 may provide an input to a remote generative machine learning model over a network, which then executes a task based on the input and returns an output to the computer 10 over the network, via the network interface 116. Alternatively, the computer 10 may instead receive input data over a network via the network interface 116 and use the input data to generate output data comprising analysis of a clinical interview. This output may then be returned to the original sender over the network. In this case, the computer 10 can effectively act as a server for performing each method.

It would be appreciated that the methods of the invention described below can be performed by any suitable computing apparatus.

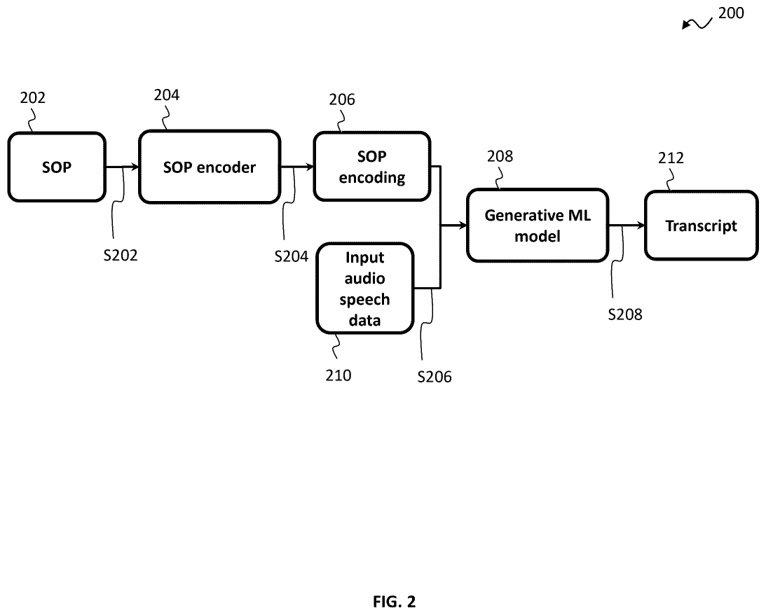

FIG. 2 shows a flowchart of a method 200 of analysing a clinical assessment to generate a transcription of an interview of the clinical assessment according to an embodiment of the invention. The method 200 can be performed by the transcription module 118 of the computer 10 to generate a transcription of a clinical interview between an interviewer and a patient. The interview can be a step in diagnosing a health condition, such as a cognitive or mental health condition.

In step S202, which is an optional preliminary step, an SOP 202 is provided to an SOP encoder 204. In the method 200, the SOP 202 is an administration SOP that provides instructions for administering a clinical interview in the form of questions and sample patient answers. Any suitable type of encoder may be used to encode the SOP 202. In one example, the encoder is a script, module or other type of software for converting a document into a structured format.

In step S204, the SOP encoder 204 encodes the SOP 202 into an SOP encoding 206, which is the SOP 202 in a particular format that allows it to be processed (or processed more effectively) by a generative ML model 208.

A simplified example of an SOP encoding 206 is provided below, where the SOP encoding 206 is in a JSON format and relates to a simple task where the patient is asked to count to five. The JSON includes an ID field for identifying the task, in this case “count_to_five”. As shown below, a single question and sample patient response is provided in this SOP encoding 206, although several questions and answers may be provided in practice. In this case, no further interviewer behaviour or action is required, therefore a corresponding field for this in the JSON is empty.

### Example SOP Encoding 1:

```
{
  “components”: [
    {
      “id”: “count_to_five”,
      “interviewer_script”: “I want you to count from one to five.”,
      “interviewer_behavior”: [ ],
      “sample_patient_responses”: [“Um. One, two, three, er, f-four,
      five.”]
    }
  ]
}
```

The sample patient response includes disfluencies, such as stuttering: “f-four” and delays: “Um”. As described further below, including this information in the sample response helps to condition the generative machine learning model 208 to provide a more clinically useful transcription of the interview.

In other examples, the SOP 202 may be pre-encoded, in which case steps S202 and S204 may be skipped.

In step S206, the SOP encoding 206 and input audio speech 210 are provided as first and second inputs, respectively, to the generative ML model 208. In the method 200, the generative ML model 208 may be an audio-to-text transformer generative ML model, such as Whisper. The SOP encoding 206 and the input audio speech 210 may be provided as different parts of an input prompt to the generative ML model 208. For example, the generative ML model 208 may be prompted to transcribe the input audio speech 210 in view of the input audio speech 210 being a recording of a clinical assessment following the SOP encoding 206.

The SOP encoding 206 is provided to condition the generative ML model 208, or, in other words, to provide context for the generative ML model 208, so that the produced output is more likely to be appropriate for the specific task. In this case, the specific task is transcription. For example, the sample answer in the example encoded SOP above includes the text “one, two”. This helps the generative ML model 208 to disambiguate between homophones “won” and “one”. In a broader sense, the encoded SOP 206 conditions the generative ML model 208 to assign higher probabilities to terms included or related to the SOP 202.

In step S208, the generative ML model 208 analyses the first and second inputs provided in the previous step and produces a transcription 212 of the input audio speech 210.

The disfluencies included in the sample patient response condition the generative ML model 208 to include, rather than filter out, disfluencies. An example output transcription 212 is: “One, t-two, er, three. Um. Four. And five”. It has been recognised that such disfluencies represent clinically relevant data for cognitive and non-cognitive health conditions. Existing transcription tools that do not condition models based on clinical SOPs or other forms of templates can filter out such disfluencies. Such approaches may interpret the same audio input speech 210 as: “Won too. Three. Four. And five”. Thus, compared to the transcript 212 produced by the method 200, the transcription of the known system is less likely to be accurate in the context of the clinical assessment task at hand (counting to five) and excludes clinically relevant information.

In one more detailed implementation, the generative ML model 208 is a Transformer which has an encoder and a decoder. The Transformer encoder takes as input sequential audio representations which are typically transformed via trained layers of attention, normalisation and feedforward neural networks. The Transformer decoder takes as input one output text token at a time and autoregressively generates the next token via trained layers of attention, normalisation, feedforward networks and additional attention over the outputs of the Transformer encoder. The input audio speech 210 can be fed into an encoder of the generative ML model 208 and the sample patient responses in the SOP encoding 206 are fed as the tokens into an autoregressive decoder of the generative ML model 208 in order to condition the decoder's output. In response, the audio-to-text model autoregressively generates a conditioned transcript, which uses both the output from the audio encoder and the conditioning textual input of the SOP fed into the decoder.

The approach of conditioning a generative machine learning model based on a template for performing a clinical interview can be extended to other types of analyses related to the interview. Further uses are set out in further methods described below.

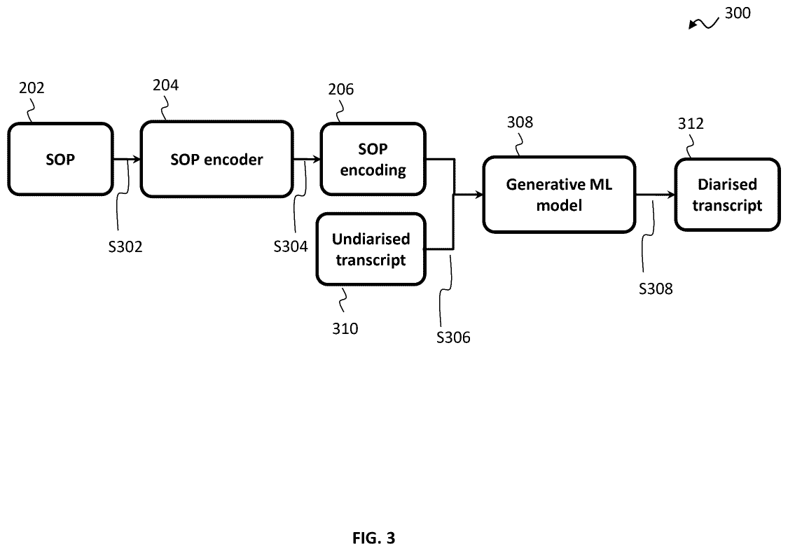

FIG. 3 shows a flowchart of a method 300 of analysing a clinical assessment to generate a diarised transcript of an interview of the clinical assessment according to an embodiment of the invention. The diarised transcript can be produced to facilitate a diagnosis of a health condition by a human or a software-based diagnosis method. “Diarisation” as used herein can also be described as speaker identification. The method 300 can be performed by the diarisation module 120 of the computer 10.

The method 300 uses a similar approach to the method 200, wherein the SOP 202 is optionally fed into an SOP encoder 204 to provide the SOP encoding 206 in steps S302 and S304, respectively. Alternatively, the SOP 202 may be pre-encoded.

The method 300 also uses the SOP 202, which is for administering a clinical assessment, and the corresponding SOP encoding 206.

In step S306, the SOP encoding 206 and an undiarised transcript 310 are provided to a generative ML model 308 for performing diarisation. These inputs can be provided as a single input prompt to the generative ML model 308.

The undiarised transcript 310 can be in an indexed format where each word has a corresponding index. An example of this is shown below.

```
“transcript”: [
  {“index”: 0, “word”: “Please”},
  {“index”: 1, “word”: “count”},
  # ...
]
```

This allows a portion of transcript to be referenced using the first and last indices. This format can also be used for the other methods described herein.

In this example, the input prompt also comprises instructions on how to format the output, for example into a JSON format. These formatting instructions can be considered as a third input. Parsing unstructured text to structured outputs may be performed using output parsing or function-calling parsing (in both cases: conditioning the generative ML model 308 to be likely to output e.g. JSON following an API schema). Requesting the output diarised transcript to be in a structured format advantageously enables the diarised transcript to be immediately processed by other functions, such as API-based web functions, or methods involving further generative models, such as the further methods described below. In other examples, the output can be a in a text string format.

Continuing with the example task described above, an example undiarised transcript 310 is: “One, t-two, er, three. Um. Four. And five”. In general, the undiarised transcript 310 can be the output transcript 212 of the method 200. The undiarised transcript 310 is a simple text string in this example.

In this example method 300, the generative ML model 308 is a text-to-text generative transformer model, such as GPT-4 chat completion. Other suitable generative models may be used in other embodiments.

In step S308, the generative ML model 308 analyses the input prompt provided at step S306 and produces a diarised transcript 312 that maps each element of the undiarised transcript 310 to one of the interviewer or the patient participating in the interview. An example diarised transcript 312, which is provided in a structured JSON format, is provided below:

```
“transcript”: [
  {
    “speaker”: “interviewer”,
    “text”: “Please count to five.”
  },
  {
    “speaker”: “patient”,
    “text”: “One, t-two, er, three. Um. Four. And five.”
  },
  [...]
]
```

The SOP encoding 206 provides context for the generative ML model 308 and conditions the output diarised transcript 312 to be more accurate for the specific purpose of diarising a clinical interview following the SOP 202. It has been found that the quality of the diarised transcript 312, that is, the accuracy of the speaker identification, is significantly improved with respect to a non-conditioned approach.

In more detail, the generative ML model 308 autoregressively generates a JSON string whose output matches the requested output schema, that was provided as a third input in step S306. The content of the SOP 202 is used to infer which words are the patient's and the interviewer's.

The methods 200 and 300 are described as separate methods, however it would be appreciated that in practice the methods 200 and 300 can be performed in sequence to produce a diarised transcript from the input audio speech 210.

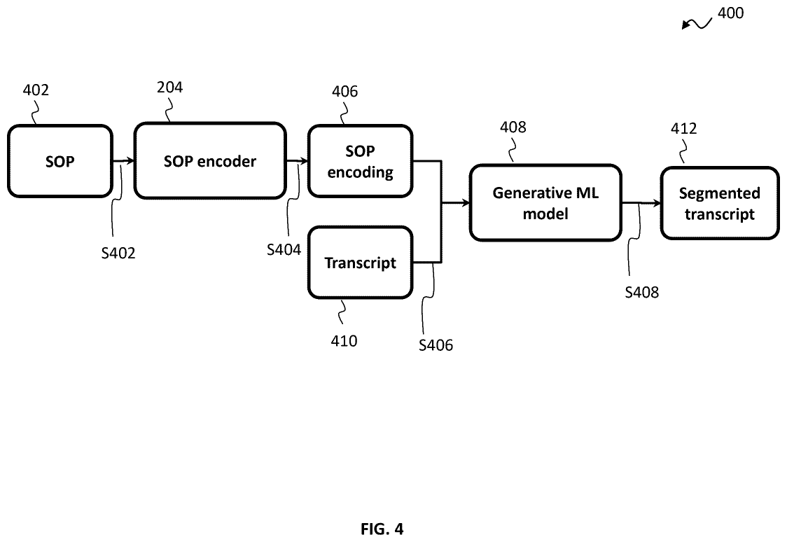

FIG. 4 shows a flowchart of a method 400 of analysing a clinical assessment to generate a segmented transcript of an interview of the clinical assessment according to an embodiment of the invention. The segmented transcript can be produced to facilitate a diagnosis of a health condition by a human or a software-based diagnosis method. The method 400 can be performed by the segmentation module 122 of the computer 10.

“Segmentation” in this context refers to the grouping or mapping of different parts of a transcription to corresponding questions and answers in template (e.g., SOP) for performing the clinical assessment. Segmentation is a laborious and error prone task when performed by human operators. Using known approaches, however, segmentation can be difficult for ML models to perform because in practice clinical interviews may not precisely follow a prescribed order. Additionally, irrelevant conversation can occur between different tests of the clinical assessment that makes it difficult to assess the boundaries of each (clinical) question and answer in a transcript. The method 400 utilises an SOP of the clinical assessment to condition a generative ML model to perform the task of segmentation more effectively in the specific context of the clinical interview taking place.

The method 400 uses a similar approach to the methods 200 and 300, wherein an SOP 402 is optionally fed into the SOP encoder 204 to provide an SOP encoding 406 in steps S402 and S404, respectively, as described previously. Alternatively, the SOP 402 may be pre-encoded.

The method 400 uses an SOP 402, which is a template for administering a clinical assessment comprising a plurality of sections corresponding to different clinical questions. A first section comprises the “count to five” test described previously. A second section comprises a story recall test, wherein the interviewer tells a patient a story then asks the patient to recall some of the story, or as many aspects of the story as possible. An example of the SOP encoding 406, which is provided in a JSON format, is provided below:

### SOP Encoding Example 2:

```
{
  “components”: [
    {
      “id”: “count_to_five”,
      “interviewer_script”: “I want you to count from one to five.”,
      “interviewer_behavior”: [ ],
      “sample_patient_responses”: [“Um. One, two, three, er, f-four, five.”],
    },
    {
      “id”: “story_recall”,
      “interviewer_script”: “I'm going to read you a little story. [...]”,
      “interviewer_behavior”: [“If the patient cannot recall the story, [...]”],
      “sample_patient_responses”: [“Um, I think this one was about [...]”]
    }
  ]
}
```

As shown, the SOP encoding 406 comprises two sections with corresponding IDs for each task (“story_recall” and “count_to_five”). The “count_to_five” task corresponds to the example described previously. The “story_recall” section comprises an interview script, not reproduced in full for brevity, for relaying a short story to a patient. An “interviewer_behaviour” sub-section includes instructions for the interviewer regarding what to do when the patient cannot recall the story. These instructions are also not reproduced in full for brevity. In other examples, several or no conditional instructions may be provided in this subsection. A sample patient response for recalling the story is also included, though in other examples no sample responses may be included, or, more preferably, several sample responses may be included.

In step S406, the SOP encoding 406 and a transcript 410 are provided to a generative ML model 408 for segmenting the transcript into component questions and answers. These inputs can be provided as a single input prompt to the generative ML model 408 as a request. As an example of such an input prompt is: “Segment the transcript [the transcript 410] into its constituent tasks. The task SOP is [SOP encoding 406] . . . ”). In this example, the input prompt also comprises instructions on how to format the output, for example into a JSON format, as described previously. For example, the input prompt can request the generative ML model 408 to segment the transcript 410, which is a transcript of a clinical interview following an SOP according to the SOP encoding 406 (as in the example 2 above), into constituent questions and answers, in the structure of JSON format [Y]. This additional input may be worded “ . . . corresponding to a list of segments, where each segment has an id (string) and a transcript, and each transcript has a list of speaker turns, where each speaker turn has a speaker label (string) and the text spoken (string)”

The transcript 410 in this example is diarised, and, more specifically, is the output diarised transcript 312 of the method 300, but may be undiarised in other examples. In general, the transcript 410 can be the output diarised transcript 312 of the method 300. The segmented transcript 412 is text in the format of a structured JSON, in this example, however the segmented transcript 412 can be output in other formats in other embodiments.

In this example method 400, the generative ML model 408 is a text-to-text generative transformer model, such as GPT-4 chat completion. Other suitable generative models may be used in other embodiments.

In step S408, the generative ML model 408 analyses the input prompt provided at step S406 and produces a segmented transcript 412 that maps each element of the transcript 410 to a particular question and answer described in the SOP 402. An example segmented transcript 412, which is provided in a structured JSON format, is provided below:

### Segmented Transcript Example:

```
{
  “segments”: [
    {
      “id”: “count_to_five”,
      “transcript”: [
        {
          “speaker”: “interviewer”,
          “text”: “Please count to five.”
        },
        {
          “speaker”: “patient”,
          “text”: “Um, one two three four five.”
        },
      ]
    },
    {
      “id”: “story_recall”,
      transcript”: [...]
    }
  ]
}
```

As shown, the segmented transcript 412 is partitioned into sections labelled with IDs corresponding to the IDs of the sections of the SOP encoding 406. The SOP encoding 406 conditions the generative ML model 408 to perform this task more effectively for the specific clinical assessment of the interview.

Having a segmented transcript enables several steps that are required for performing automated diagnosis to be performed more effectively. In particular, automated evaluation or (“rating”) of a clinical test and automated quality control review of the evaluation can be performed much more effectively for input data that has been segmented into component questions and answers. It has been found that the quality of the segmented transcript 412, that is, the accuracy of the question/answer identification, is significantly improved with respect to a non-conditioned ML approach.

In more detail, the SOP encoding 406 may be passed into a system message of the generative ML model 408 together with the input prompt. The input transcript 410 can be passed directly into the user message of the system in a JSON-encoded format. The generative ML model 408 autoregressively generates an output that matches the schema provided in the input prompt, restructuring the input transcript 410 and conditioned by the SOP encoding 406. The segmented transcript 412 may be generated in a system message.

As an aside, “system” and “user” messages are roles framing input prompts specific to some models. Models like GPT-4 are based on ‘chat’ style data: predicting the next interaction given a conversation history. The roles can be anything but are typically ‘user’, ‘assistant’ and ‘system’. There is no fundamental difference between the content of, e.g., system and user messages, though they can be designed to perform different functions. During training, ‘assistant’ can be been used to represent the machine learning model's response, ‘user’ can be used to represent the human's response, and ‘system’ can be used (typically at the start) to influence (condition) the behaviour of the machine learning model. In effect, ‘system’ becomes a conditioning message that influences the ‘assistant's’ behaviour.

As an optional further step, an audio recording of the interview can also be segmented, based on the segmented transcript 412, to produce a segmented audio, which may be useful for some further applications. In one example, the segmented transcript 412 may include timestamps, and the segmented audio may be produced using the timestamps.

The methods 200, 300 and 400 are described as separate methods, however it would be appreciated that in practice these methods can be performed in sequence to produce a segmented transcript from the input audio speech 210.

As already mentioned, the segmented transcript 412 enables automated rating, or “evaluation”, of a clinical interview to be performed more effectively.

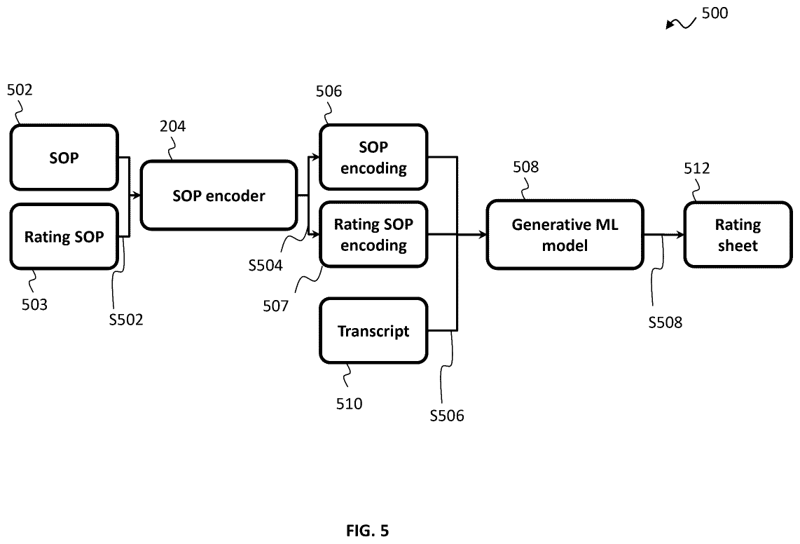

FIG. 5 shows a flowchart of a method 500 of analysing an interview of a clinical assessment to generate a rating or evaluation for the clinical assessment according to an embodiment of the invention. The rating or evaluation may represent a likelihood that the patient has a particular health condition. Alternatively, the rating or evaluation can be a factor of a larger decision-making process in a diagnosis of a health condition by a human or a software-based diagnosis method. For example, the method 500 may be used to automatically score one or more tests in a clinical assessment, for a human or computer-led diagnosis to be made based on the score. Thus, the method 500 can be part of a process in making a diagnosis of a health condition.

The method 500 can be performed by the rating module 124 of the computer 10.

The method 500 uses a similar approach to the methods 200, 300 and 400, wherein an SOP 502 is optionally fed into the SOP encoder 204 to provide an SOP encoding 506 in steps S502 and S504, respectively, as described previously. The SOP 502 is identical to the SOP 402 and provides instructions for administering the story recall test; however, the SOP 502 is limited to the section for the story recall test only, for the purposes of illustration. Alternatively, the SOP 502 may be pre-encoded.

In the method 500, a rating SOP 503 is also optionally encoded by the SOP encoder 204 into a rating SOP encoding 507 in steps S502 and S504, respectively. Alternatively, the rating SOP 502 may be pre-encoded. The rating SOP 502 is a template for evaluating the interview that can be used by a clinician to score the patient's answers to the questions of the clinical assessment. An example of the rating SOP encoding 507 in JSON format is provided below:

```
{
  “instructions”: “Decide whether each story element was recalled. Allow
paraphrases.”,
  “story_elements”: [
    {
        “element”: “Allison”,
        “scoring_guidance”: “Accept only Allison.”
    },
    [...]
  ],
  “output_schema”: [
    “element_idx”: {
      “type”: “int”,
      “description”: “The index of the story element.”,
    },
    “recalled”: {
      “type”: “bool”,
      “description”: “Whether this element was recalled.”
    }
  ]
}
```

As shown, the rating SOP encoding 507 comprises an instructions sub-section and a text string entry providing instructions for scoring the task. The instructions can include guidance such as “allow paraphrases”. A “story_elements” subsection is provided that lists each aspect of the story. The example above has been limited to a single story element (“Allison”) for simplicity, though it would be appreciated that further elements may be provided in practice. Guidance for scoring the particular story element is also provided in the form of a “scoring_guidance” subsection. An “output_schema” subsection is included to provide a format for the story element. The output schema conditions the model to provide its output in a particular format. In this case, ‘rating’ the story recall means producing a report that includes the element index and whether or not that element was recalled. A schema, or template, has children (a type and description) that tells you (a) what kind of data should be populated in this field, and (b) a description of what the field means. A “recalled” section provides a description for the evaluation (“whether this element was recalled”) and a type of the evaluation (“bool”). In other examples, the evaluation could be measured in other ways, such using a scale of 1 to 10, which may be reflected in the “type” subsection.

Other sub-sections or sections may be provided in other examples. For instance, a sample completed worksheet showing the results of one clinician's evaluation of a different sample interview could be included in the rating SOP 503.

It would be appreciated that the content of the rating SOP 503 and the corresponding rating SOP encoding 507 would vary significantly depending on the particular health condition the clinical assessment seeks to diagnose.

In step S506, the SOP encoding 506 and the rating SOP encoding 507 are provided as inputs to a generative ML model 508 along with a transcript 510.

In this instance, the transcript 510 is the segmented transcript 412 is the output of the method 400. However, the transcript 510 could be a transcript produced by other methods or means. The transcript 510 is preferably diarised, segmented, and in a structured format, for best results.

In this example method 500, the generative ML model 508 is a text-to-text generative transformer ML model, such as GPT-4 chat completion. Other suitable generative ML models may be used in other embodiments.

As before, these inputs can be provided as inputs to the model in a prompt, optionally with instructions to format the output in a structured form, such as JSON or XML. The generative ML model 508 may be prompted to provide the output in the form of a “rating sheet”, although the output could be in various forms of natural language or structured text.

In step S508, the generative ML model analyses the provided inputs and provides an output rating sheet 512. An example encoding of the rating sheet 512 is provided below in a JSON format:

### Rater Sheet:

```
{
  “output”: [
    {
      “element_idx”: 0,
      “recalled”: true,
    },
    {
      “element_idx”: 1,
      “recalled”: false,
    }
  ]
}
```

In this example, the rating sheet comprises a series of subsections corresponding to each story element with Boolean indication of whether the story element was present in the patient's response. Each story element is indexed in a manner matching the indexing described in the rating SOP encoding 507. The example above shows that a first story element (corresponding to index 0) was present and a second story element (index 1) was not present.

The output of the generative ML model 508 in the method 500 could be formatted in various other ways depending on the intended downstream use of the rating. Providing a structured output format, however, means the output is already in a format that can be processed efficiently by other computing systems. For example, the rating sheet 512 can be passed on to a web-interface that presents the results of the story recall test to a clinician. It is worth noting that interviews for these tests can last several hours. The method 500 can automatically review and score these tests, meaning a human clinician is not required to manually listen to and score the story recall test. Thus, analysis provided by the method 500 can save a significant amount of time for the clinician, enabling a diagnosis to be made more quickly and freeing up the clinician's time to perform other tasks.

Furthermore, the corresponding encodings of the (administration) SOP 502 and the rating SOP 503 condition the generative ML model 508 to perform the rating task more effectively in a similar manner as described above.

In more detail, SOP encodings 506, 507 are passed into a system message of the generative ML model 508 with task instructions (“Rate the task response as per the rating SOP encoding. The administration SOP is [SOP encoding 506]. The rating SOP is [rating SOP encoding 507]”). The transcript 510 can be directly passed into a user message of the generative ML model 508, which then autoregressively generates a JSON matching the rating worksheet schema by parsing the transcript 510, conditioned by the SOPs in the system message.

The rating SOP encoding 507 can include instructions on how to interpret or adjust an evaluation based on disfluencies of the patient. Alternatively, or in addition, the generative ML model 508 can be trained with positive training data corresponding to patients diagnosed with the health condition in question, in order assess the relevance of disfluencies (or other speech features) to each criteria in the SOP 502. When combined with a high-quality transcription obtainable using the method 200, this allows the rating performed by the method 500 to factor-in clinically relevant information into the evaluation that would be very difficult or impossible for a human operator to account for.

The methods 200, 300, 400 and 500 are described as separate methods, however it would be appreciated that in practice these methods can be performed in sequence to produce a rating sheet 512 from the input audio speech 210.

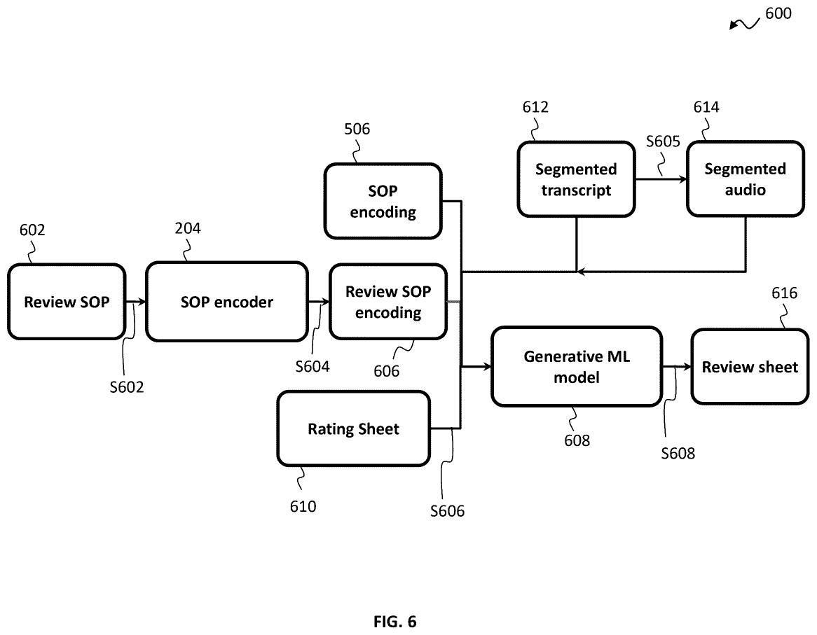

Conditioning of ML models in this manner can also be used to quality check the administration and/or rating of a clinical test. A method 600 for this purpose is shown in FIG. 6.

FIG. 6 shows a flowchart of a method 600 of analysing an interview of a clinical assessment to perform a quality-check or “review” of an evaluation of a clinical assessment according to an embodiment of the invention. The evaluation can be the kind of rating or evaluation that is the output of the method 500, such as a completed rating sheet. The evaluation can be completed by human or computer-implemented method. The method 600 can be used as part of a clinical assessment and may also be used as part of a diagnosis of a health condition. The method 600 can be performed by the review module 126 of the computer 10, in one example.

The method 600 uses a similar approach to the methods described previously, wherein a review SOP 602 is optionally fed into the SOP encoder 204 to provide a review SOP encoding 606 in steps S602 and S604, respectively. The review SOP 602 comprises instructions for checking or verifying various aspects of the administration and/or rating of a clinical assessment. In the review SOP encoding 606, these instructions or guidance can be encoded in a structured format, such as a JSON format having a similar structure to the example rating SOP encoding shown above. Several sections may be included in the review SOP encoding 606, each with instructions for reviewing a specific clinical test outlined in an SOP encoding for administering the clinical assessment.

The review SOP encoding 606 can include an API schema, enabling the generative ML model 608 to autoregressively generate an output comprising a structured JSON matching the review worksheet API schema.

An SOP encoding for administering the clinical assessment is also provided as a further input to provide further context for the review. In this example, the SOP encoding 506 is also provided as a further input, though it would be appreciated that other SOP encodings with several sections could be provided in other embodiments. As before, some embodiments may include a preliminary step of performing the encoding of the SOP encoding 506.

In step S605, a segmented transcript 612 is optionally used to produce corresponding segmented audio 614 of a recording of the interview, for example using timestamps in the segmented transcript 612. The segmented transcript 612 can be produced using the method 400, in one example. For best results, the segmented transcript 612 is also diarised and includes disfluencies of the patient.

In step S606, the SOP encoding 506, the review SOP encoding 606, the segmented transcript 612, and the segmented audio 614 are provided as inputs to a generative ML model 608 together with a rating sheet 610. As before, these inputs can be provided as inputs to the model in a prompt, optionally with instructions to format the output in a structured form, such as JSON or XML.

In this example, the rating sheet 610 is of a similar format and structure to the rating sheet 512 of the method 500. However, in general, the rating sheet 610 can be any set of criteria that has been evaluated as part of a clinical assessment, in natural language or structured formatting. In this example, the method 600 reviews both the evaluation and the administration of the clinical assessment, and therefore includes the rating sheet 610. In other examples, the rating sheet 610 may not be necessary when the method 600 is performed only to review the administration of a clinical assessment.

In other examples, only one of the segmented audio 614 or the segmented transcript 612 may be provided. Non-segmented transcripts may be provided in other examples, however it has been found that diarised, segmented transcripts produce the best results.

In this example, the generative ML model 608 is same type of model as the generative ML model 508. Other suitable models may be used in other example embodiments.

Full (i.e., for the whole clinical assessment) SOPs, transcriptions and a full rating sheet can be provided as an input the generative ML model 608 in step S606. Alternatively, because both transcripts 612, 614 are segmented, and the SOPs 506, 606 and rating sheet 610 can comprise corresponding sections, these inputs can be provided to the generative ML model 608 in chunks corresponding to a particular question/answer or a particular part of the clinical assessment. In this case, the method 600 may be performed iteratively, based on successive chunks of the clinical assessment, until all of the clinical assessment has been reviewed. Generative ML models can produce better results for more concise input data. Therefore, performing the method 600 iteratively in this way can produce better results.

It would be appreciated that the previous methods 200-500 can also be performed iteratively in the same way.

In step S608, the generative ML model 608 analyses the inputs provided in step S606 and produces, as an output, a review sheet 616. The review sheet 616 may be completed or a part of a complete review sheet, depending on whether the method 600 is performed iteratively. The review sheet 616 can be in a structured format, as described with previous outputs.

Similar to the method 500, reviewing a clinical assessment in this way saves a significant amount of time for human clinicians. The generative ML model 608 is conditioned based on the SOP encoding 506 and the review SOP encoding 606 to perform the reviewing task more effectively for the context of the particular clinical assessment under review.

The method 600 is ideally performed together with the methods 200, 300 and 400 to perform a review of a clinical assessment based on an audio recording of an interview of the clinical assessment together with an evaluation of the assessment, such as a rating sheet. This saves a significant amount of time in total. Additionally, the transcriptions can include patient disfluencies picked up by the method 200, so that more clinically relevant information can be analysed. The method 600 could also be performed on a rating produced according to the method 500.

As discussed above, conditioning a ML model in the manner of the present invention can be performed in “chunks” corresponding to single questions and answers between a patient and clinician in a clinical interview. This approach can also be extended to automatically administer a clinical assessment in real time.

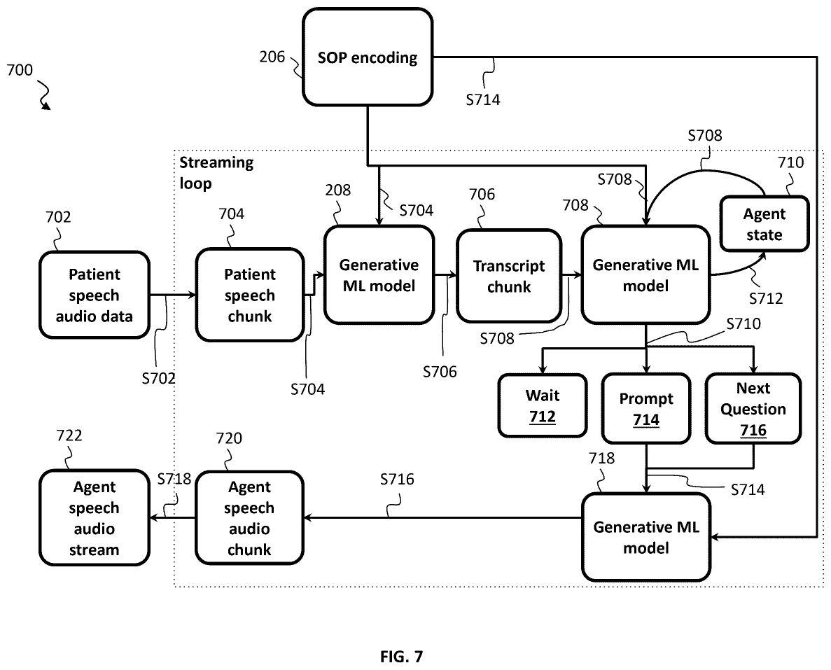

FIG. 7 shows a flowchart of an iterative real-time method 700 of administering a clinical assessment by analysing chunks of patient speech and providing an appropriate response. The method 700 can be performed by the administration module 127 of the computer 10, in one example. Any speech-based clinical assessment can be administered using the method 700.

In step S702, a stream of audio data 702 representing patient speech is received. This can be recorded directly by the audio equipment 128 or received remotely through the network interface 116. The audio data 702 is continuously split into patient speech chunks 704 as part of a streaming loop indicated by the dashed box. Gaps or pauses in the patient's speech can be used to partition each speech chunk 704. Alternatively, each patient speech chunk 704 may be a set duration.

In step S704, a patient speech chunk 704 is provided to the generative ML model 208 together with the SOP encoding 206. This can be performed as described in the method 200 above. As before, a preliminary step of encoding the SOP 202 can also be performed in some embodiments. In step S706, as described above in relation to the method 200, the generative ML model 208 produces conditioned a transcript chunk 706 corresponding to the patient speech chunk 704.

In step S708, the transcript chunk 706 is provided as an input to a text-to-text generative ML model 708 for administering the clinical assessment. The SOP encoding 206 is also provided as a further input to condition the generative ML model 708 to the particular context of the clinical assessment.

A further input is provided at this step, namely an agent state 710 representing a current status of the interview. The agent state 710 can include a previous conversational history of the interview, an indication of the current test or section of the clinical assessment that is under way, or any other information relevant to the status of the ongoing interview. The agent state 710 is updated between iterations of the streaming loop to track the progress of the interview.

In step S710, the generative ML model 708 analyses the inputs provided in step S708 and selects an action to be performed. The action can be any of a large number of possible actions. One option is a wait action 712 that can be selected when the generative ML model 708 determines the patient is still talking. A further option is a prompt action 714 to prompt the patient for more information on a topic. A next question action 716 could be selected when the generative ML model 708 has established the current question has been answered.

Other actions could be implemented, such as an action for calling for human assistance when patient distress is identified. In yet further examples, the possible actions could include deciding to call an external API or a web-hook, and/or updating the agent state 710 with either external parameters (e.g. using a task sequence/policy) or parameters that the model 708 generates (conditioned by the inputs and agent state 710).

In this example, these output actions are embodied as a text output in a structured format, comprising an indication of the selected action as well as generated natural language that should be relayed to the patient. These can be provided in any suitable structured format and may be similar to the examples above. The structured output can encode the name of the action to call and any relevant parameters to pass onwards. The selected action could be embodied in other ways in other examples.

In step S712, the generative ML model 708 also generates an updated agent state 710 to record the progression of the interview so that subsequent real-time iterations of the method 700 can select an appropriate action. For instance, the new agent state 710 may reflect that the interview has moved onto a different question in the SOP encoding 206. Thus, the agent state 710 is preserved between streaming loop iterations.

The updated agent state 710 may not be distinct from the text output of step S710. For example, an index or a Boolean indication in the structured output of step S710 can indicate the stage or progression of the interview, which is noted by the administration module 127.

In the case where a wait action 712 is selected, an iteration of the method 700 is completed and a further patient speech chunk can be processed in the same manner. However, further steps can occur if a non-passive action is selected.

In step S714, for instance, natural language generated for the selected action can, if present, be provided as an input to a text-to-speech generative ML model 718 for generating speech signals to be relayed to the patient. In this example, the prompt action 714 is selected at step S710 and the generative ML model 708 generates natural language text in its output for asking the patient to expand on part of their response provided in the most recent transcript chunk 706.

In S714, the SOP encoding 206 is also provided as an input to the generative ML model 718 to condition the generative ML model 718 to provide a more appropriate output in the current clinical context. The SOP encoding 206 could be used by the generative ML model as context to disambiguate heterophones (two words with the same spelling but different meanings and pronunciations) in the generated natural language, for example.

In step S716, the generative ML model 718 analyses the inputs provided at step S714 and generates an agent speech audio chunk 720. In this example iteration, the audio chunk 720 comprises an audio signal that, when played by a playback device, sounds like a human operator asking the patient for further information on a topic.

In step S718, the audio chunk 720 is relayed to a speaker in the vicinity of the patient via the network interface 116 or directly to audio equipment 128 to form an audio stream 722. The speaker continuously plays the audio stream 722 in each iteration of the method 700 in real time to deliver the clinical assessment.

At step S718, a status check can be performed to determine whether all questions in the SOP encoding 206 have been asked and answered adequately. If not, the method 700 can return to step S702 and iterate until the full assessment outlined in the SOP encoding 206 has been completed.

The method 700 can be considered as operating as a ‘state machine’. That is, the generative ML model 708 decides at each step whether to e.g. continue prompting or move onto the next question as per the SOP encoding 206; this decision updates the agent state 710 of the model and allows it to sequentially iterate through the SOP encoding 206.

The methods 300 and 400 can be performed after the completed interview to diarise and segment the interview, respectively. Alternatively, it may be possible to diarise and segment the interview without using these methods, using timestamps noted during the method 700, corresponding audio or transcript chunks and corresponding SOP sections.

A resulting segmented and diarised transcript can be used as an input to the method 500 for rating the interview after the interview has been completed with the method 700. This allows the clinical assessment carried out by the method 700 to be evaluated as described previously, for instance to determine a score for the assessment or make one of a positive or negative diagnosis of a health condition. The method 600 can be performed afterwards to review the evaluation according to review criteria in a review SOP.

Alternatively, a diarised and segmented transcript of the completed interview can be passed to a human clinician to perform a diagnosis.

Administering clinical assessments in this manner can serve patients at a much higher rate than human operators to offset shortages in human specialists. The method 700 can be performed partially remotely over a network, allowing data collection and in some cases diagnosis to be performed in any geographical location suffering from a shortage of relevant specialists.

The conditioning utilised by the methods above can invoke so-called “zero” or “few-shot transfer learning” of large language models, which is a phenomenon in which large language models (such as GPT-4) were found to be proficient at performing tasks outside of their training set. This means that little or no specific training is required for the generative models to perform the various tasks discussed above. For this reason, general-purpose models can be used in the methods described herein, as well as more specialised or human-trained models. Further, this allows the methods to be used for many different kinds of speech-based clinical assessments without requiring specific training.

In the above example, the text-to-speech generative ML model 718 is used to generate sound signals that are replayed to the patient. However, it would also be appreciated that non-machine learning model based methods could be implemented in other embodiments to generate speech from text. In addition or alternatively to generating the audio stream 722, the natural language of each selected action by the generative ML model 708 can be shown to the user on a graphical user interface, e.g., to support patients who are hard of hearing.

The example tasks set by the interviewer in the example embodiments described above are predominantly speech-based tasks (i.e., questions) for the purposes of illustration. In other embodiments of the methods 200-700, the input data can include any type of data recorded during a clinical assessment. In one example, the tasks set by the interviewer can include non-speech-based tasks, where the patient's answers to these tasks are recorded during the interview by video or image-capturing equipment. For instance, the patient could be shown a drawing, then asked to re-create the drawing from memory. Similarly, patients may be asked to perform physical tasks, which are recorded by video equipment during the clinical assessment. Video or image recordings of these alternative types of tasks can be provided as inputs during, for example, the method 400, to determine which task set by the interviewer the recording(s) correspond to. These alternative tasks could also be provided as inputs in the methods 500 and 600 for rating and reviewing the clinical assessment, respectively, or received as an input in the method 700 for administering a clinical assessment.

Moreover, the methods 200-700 above have been explained as including a patient and an interviewer as the only participants of the clinical assessment. However, the clinical assessment can also include an “informant” for the patient, who can be a relative or caregiver that can act when required as an intermediary between the patient and interviewer. The methods 200 and 300, for example, may include relevant information about informants in the SOP 202, to condition the respective machine learning model to transcribe or diarised the informant's speech more effectively. Informants are common in Clinical Dementia Rating clinical assessments.

In the various methods described above, it has been found that using complete input data and SOPs that cover the whole of a given clinical assessment can, in some cases, exceed context or prompt limits for some machine learning models. In other cases where the context limits have not been exceeded, it has been found that better results can be obtained by splitting the input data (of any type, e.g., text, audio, video, or image data) and the SOPs into smaller chunks.

More particularly, it has been recognised that splitting the input data and SOPs into corresponding “segments” and providing these corresponding segments as the inputs to the machine learning models described above produces particularly good results. Surprisingly, each segment of the SOP provides sufficient context to condition the machine learning model to analyse the corresponding segment of the input data more effectively in the context of the clinical assessment. Splitting the SOPs and input data in this way can be referred to as “pre-segmenting” the inputs of the machine learning model.

A “segment” of an SOP can be a single question or task that is set by an interviewer in a clinical assessment. In some cases, a segment in an SOP could also be a group of questions related to a single clinical topic.

Taking the specific example of the method 200, the SOP 202 for administering a clinical assessment can include several distinct segments aimed at assessing different aspects of the patient's condition. Each segment can include several interviewer questions or tasks, or may be limited to a single question or task. A clinician may be able to provide an overall rating for each segment, as well as individual ratings for each question or task in the segment. Following this approach, the method 200 can be performed repeatedly to produce a transcript over several iterations. In each iteration, a part of the transcript 212 corresponding to a specific segment is output by the generative ML model 208, based on inputs comprising a corresponding segment of the SOP 202 and a corresponding segment of the input audio speech data 210. This can produce a more or equally accurate transcript compared to performing the method 200 in a single iteration, while ensuring the inputs to the generative ML model 208 can fit within a maximum context data limit. It would be appreciated that the methods 300-600 can be performed iteratively using pre-segmented input data in the same way. The method 700 may not require pre-processing in this manner, since it already functions iteratively in a streaming loop.

Manually separating input data into segments corresponding to SOP segments is time consuming. Therefore, a computer-implemented method is needed for performing this process. One challenge with performing this pre-segmentation in an automated manner is that clinical interviews in practice tend to progress through a corresponding SOP non-linearly. This makes it difficult to partition any data recorded during the interview neatly into portions that match the SOP. A further complication is that irrelevant dialogue (“chit chat”) can occur during or between tasks. The method 800 below seeks to address these issues.

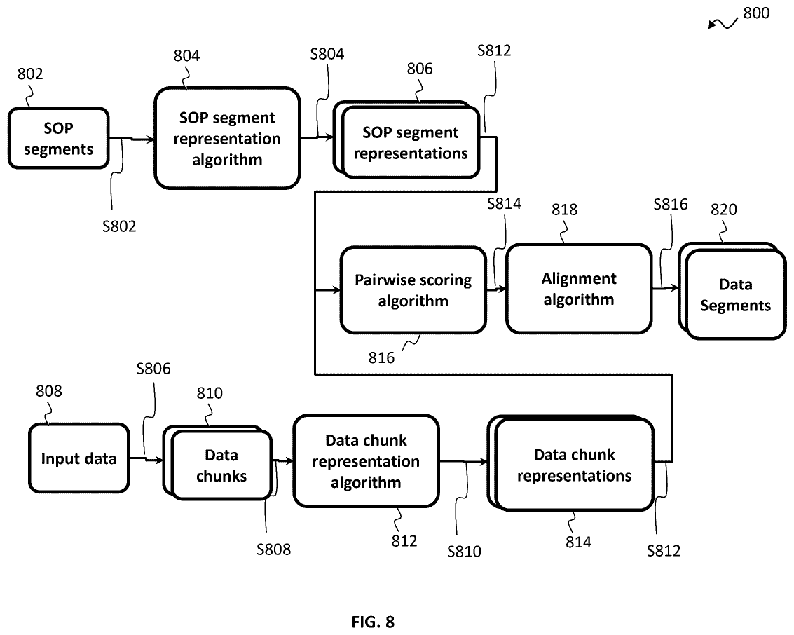

FIG. 8 shows a method 800 for splitting data from a clinical assessment into segments following the structure of a template (e.g., SOP) for administering the clinical assessment according to an embodiment of the invention.

The method 800 can be used as a pre-processing step for SOPs and data recordings from a clinical assessment that are to be used as inputs to a machine learning model in any of the methods described previously. The method 800 can be performed by the segmentation module 122 or by another module of the computer 10 specifically adapted for pre-processing assessment data.

In step S802, SOP segments 802 are optionally provided as an input to an SOP segment representation algorithm 804.

SOP segments 802 are parts of an SOP corresponding to different questions or sections of a clinical assessment. The SOP segments 802 are segments of an SOP for administering a clinical assessment in this example, but could be for other purposes in other examples, such as for rating or reviewing a clinical assessment, as described previously. The SOP segments 802 can be comprised of natural language text and/or may be in a structured format, such as JSON, XML, Markdown, HTML, TypeScript, etc, as described previously. The SOP segments 802 could be manually partitioned from an SOP into different segments. More preferably, an SOP in a structured format could include an index for each section or question. Each interviewer question and any sample patient responses are each grouped by a particular index. This would enable straightforward and automatic splitting of an SOP into several structured format objects to form SOP segments.

The SOP segment representation algorithm 804 converts each of the SOP segments 802 into an embedding to enable semantic comparison with portions of the input data 808. “Embedding” refers to encoding in a vector space, as known in the art, however, embedding the SOP segments 802 is not required. The segment representation algorithm 804 may produce one or more of: contextual word embeddings from a language model; non-contextual word embeddings from e.g. a bag-of-words model; audio, linguistic or audio-linguistic embeddings; summary produced by a generative model; simulated audio-linguistic data produced by a generative model. The SOP segment representation algorithm may selectively use one or more components of the SOP, for example: the interviewer script, the example patient responses; or a query generated from either the interviewer script and/or example patient responses, e.g. by a machine learning model. For example a generative machine learning model may be used to generate a query based on a components of the SOP, such as the interviewer script or example patient responses, and the query may be encoded into an embedding.

In step S804, the SOP segment representation algorithm 804 outputs SOP segment representations 806, which are the SOP segments 802 transformed into a suitable embedded format suitable for being used as an input in subsequent processing.

In other example embodiments, the SOP segments 802 may already be provided in a format suitable for subsequent processing, and thus steps S802 and S804 may not be necessary. In this case, the SOP segments 802 may be used in subsequent steps in place of the SOP segment representations 806.

In step S806, input data 808 is split into data chunks 810.

The input data 808 can comprise any data recorded during a clinical assessment (“assessment data”), such as text, audio, or video recordings of an interview of the clinical assessment, as well as image or video data of patient responses to physical or drawing-based tasks in the clinical assessment. The input data 808 can comprise more than one type of data, for example a text transcript of a clinical interview and a corresponding audio and/or video file, for example. It would be appreciated the form of the input data 808 may depend on the type of task (e.g., which of the methods 200-600) for which the method 800 is being used to pre-segment the input data and SOPs.

In the case where the input data 808 comprises text (e.g., to perform pre-segmentation of a transcript to be used as input to the method 300 to diarise the transcript), the input data 808 may be split into data chunks 810, each data chunk comprising a window of a certain number of words in length, for example 200 words. The window size may be chosen such that a window is large enough to cover the administration instructions for any task in the corresponding SOP. Each subsequent data chunk may partially overlap with a previous data chunk by a particular stride length, such as 100 words. This can be referred to as using “strided contiguous windows”. Thus, in a simplified example of text input data comprising a total of 500 words, the input data 808 can be split into a first chunk of 200 words, a second chunk of 200 words wherein the former 100 words are the same as the latter 100 words of the first chunk, and so on, producing a total of four contiguous strided chunks of 200 words. Audio and video data may be split in a similar manner based on a time interval window and stride length, such as 10 and 5 seconds respectively. Image data in the input data 808 may not require splitting and thus could be omitted from this step. Splitting the input data 808 in this overlapping manner helps to counteract the possibility of artificially dividing patient responses (or interviewer questions, for example) in the input data 808 across different data chunks 810.

In step S808, the data chunks 810 are optionally provided as an input to a data chunk representation algorithm 812 to transform each data chunk into a format suitable for use as an input in subsequent processing. Alternatively, the data chunks 810 may already be in a suitable format for subsequent steps. In this case, subsequent steps may use the data chunks 810 in place of data chunk representations 814.

The data chunk representation algorithm 812 can be any model usable to embed the data chunks 810 into a vector space to enable semantic comparison with the SOP segment representations 806. The data chunk representation algorithm 812 may produce contextual word embeddings from a language model, non-contextual word embeddings from e.g. a bag-of-words model, speech audio representations from a deep learning model, audio-linguistic representations, among other things. The data chunk representation algorithm 812 may encode all or one or more components of the data chunk. In the case of text data, the data chunk representation algorithm selectively use one or more of: the entire undiarised transcript for the data chunk, the diarised transcript for this data chunk, the interviewer's words for the data chunk, the patient's words for the data chunk.

In step S810, the data chunk representation algorithm 812 produces data chunk representations 814 embedded into a vector space as an output.

In step S812 the SOP segment representations 806 and the data chunk representations 814 are provided to a pairwise scoring algorithm 816.

The pairwise scoring algorithm 816 compares each of the SOP segment representations 806 with each of the data chunk representations 814 to evaluate the similarity within each respective pairing. The pairwise scoring algorithm 816 produces a set of numerical representations corresponding to the similarity between a data chunk and an SOP segment. There may be multiple scores per pair.

In one example, the pairwise scoring algorithm 816 may consider every possible pairing of S SOP segments representations 816 and T data chunk representations 814 for M different representation modalities, producing a 3D tensor of dimensionality (S, T, M). “Modalities” refers to different formats of input data 808, which could be text, audio, video, or image formats. The pairwise scoring algorithm 816 can be configured to produce a pairwise cosine similarity and/or word error rate, to name some specific suitable parameters, between SOP segment representations 806 and data chunk representations 814 at each point in the tensor. Alternatively, the tensor can be a matrix of dimensionality (S, T) where only one modality is considered.

The pairwise scoring algorithm 816 can be any type of pairwise scoring algorithm suitable for determining a similarity between the input SOP segment representation 806 and the data chunk representation 814, as known in the art.

In step S814, the pairwise scoring algorithm 816 calculates pairwise scores for each pair of SOP segment representations 806 and data chunk representations 814 as an output. This output is then passed to an alignment algorithm 818.

The alignment algorithm 818 is performed to find an optimal alignment between the SOP segments 802 and the data chunks 810. The alignment algorithm 818 is required because, in practice, clinical interviews may not follow a given SOP linearly and may include some irrelevant conversation interspersed between clinical questions. It is possible that patients and clinicians could also return to previous clinical questions at a later stage in an interview. The alignment algorithm is used to restructure the data chunks 810 into an order that matches the SOP comprised of the SOP segments 802.

In step S816, once the data chunk representations 814 have been matched and ordered to a corresponding SOP segment representation 806, the alignment algorithm 818 forms, as an output, a plurality of data segments 820 from data chunks 810. Each of the data segments 820 can comprise one or several of the data chunks 810, which in general can be in a different order to the order in which the data chunks 810 are present in the input data 808. Each of the data segments 820 is matched to at least one corresponding SOP segment representations 806. It is possible to match a data segment 820 to more than one SOP segment representations 806 to account for the possibility of overlapping semantic content of data chunk representations 814.

Once this has been completed, each SOP segment 802 and the corresponding data segment(s) 820 can be provided as inputs in any of the methods 200-600 described above. These inputs are of a smaller data size that fits within certain context size limits of generative ML models that are too small to accommodate an entire SOP and interview recording. In other cases, using data segments and corresponding segments of SOPs as generated by the method 800 can produce more accurate outputs. The methods 200-600 can then be repeated iteratively for subsequent pair of matched SOP segment 802 and corresponding data segment(s) 820 until an entire clinical assessment has been analysed.

The following passages describe the operation of the alignment algorithm 818 in greater detail.

The alignment algorithm 818 considers the pairwise scores and optionally the relative (original) position or order of the SOP segment representations 806 and data chunk representations 814. The alignment algorithm 818 may find the optimal alignment by finding the path through the 3D tensor from s=0, t=0 to s=S−1, t=T−1 that maximises the sum of the scores subject to one or more constraints, along one or more dimensions. One or more of the following constraints can be used: (1) SOP segment representations 806 may not overlap in “data chunk space” (i.e. the same data chunk 810 cannot belong to two SOP segments 802), (2) SOP segment representations may not overlap more than one span (i.e. the span of one window); (3) each SOP segment representation 806 may occupy exactly one contiguous data chunk representation span (or the fewest possible data chunk spans); (4) SOP segments 802 must occur in the data chunks strictly or approximately in the order in the SOP.

The above constraints are useful because any data chunk span (i.e. assessment data split up into segments) can correspond to one or more SOP segments 802. For example, when assessment data for an SOP with 3 tasks is split into two data chunks, the first half may include 2 SOP segments inside it (task 1 and part of task 2). Similarly, the second half may include 2 SOP segments inside it (part of task 2 and task 3). Thus, SOP segments can ‘occur’ or be related partially to a data chunk. The above constraints are useful to avoid edge effects that could occur by splitting SOPs segments 802 across two data chunks, which could confuse the output of a machine learning model. Therefore, the data chunks are split into ‘sliding windows’ e.g. words 1-50, 25-75, 50-100, 75-125 etc, which overlap and constrained as above.

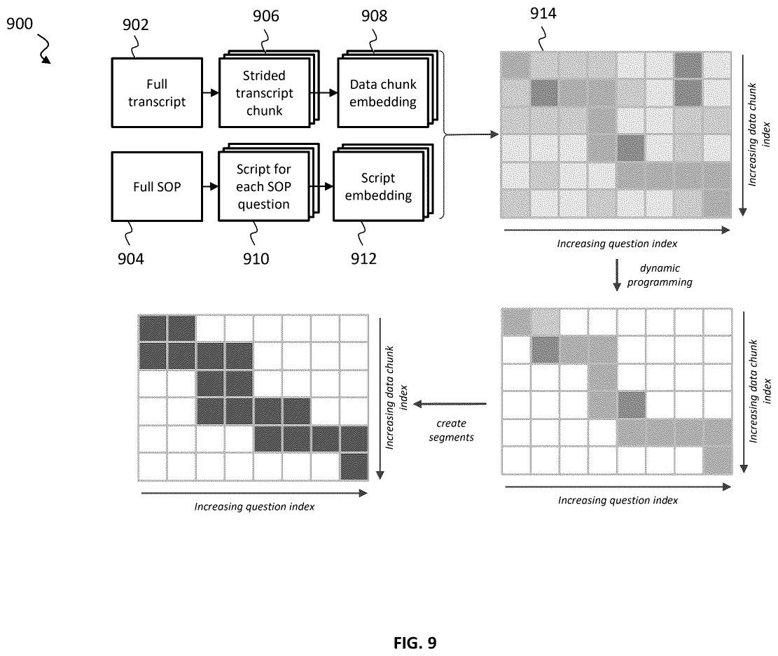

The alignment algorithm 818 may solve the optimisation problem above using dynamic programming to find the ‘maximum falling path’ from the top-left cell in the tensor to the bottom-right cell using only right-moves and down-moves. This imposes the following constraints: (i) the SOP segment representations 806 must occur strictly in the order of the corresponding SOP (this has the effect of assuming the clinical assessment happens in the order of the SOP); (ii) each SOP segment representation 806 may occupy exactly one contiguous data chunk span; (iii) SOP segment representations 806 may only overlap one data chunk (which can be implemented by selecting a sufficiently small window size to ensure a given data chunk 810 could only relate to at most two SOP segments 802). A specific example of this is described further below with reference to FIG. 9.

Alternatively, the alignment algorithm 818 may find the optimal alignment by greedy matching, such each best match between data chunk representations 814 and SOP segment representations 806 are assigned to one another, then removed from further consideration.

Alternatively, the alignment algorithm 818 may find the optimal alignment by simple thresholding, such that the possibly non-contiguous matching data chunk representations 814 and SOP segment representations 806 are determined by one or more cutoff thresholds in the pairwise similarity score or scores. In one example, the threshold can be a cosine similarity greater than 0.7 and/or a word error rate less than 0.2.

The alignment algorithm 818 may have a further processing step in which one or more “padding” chunks are added to each side of the identified chunks for a given segment to avoid windowing effects (i.e. in case one or more words of the administration fall outside the best matching chunk).

The alignment algorithm 818 may have a further processing step in which any unassigned data chunk representations 814 are assigned to the same segment as the last assigned data chunk representation 814. For example, when the SOP of the SOP segments 802 is based on an interviewer's script, unassigned words after a data chunk representation 814 determined to correspond to a particular interviewer question are likely to be the patient's response and can be added to the same data segment.

The data segments 820 produced as the output of the alignment algorithm 818 can be in various forms. In one example, the data segments 820 can be in the same format as the input data 808 but reordered to match the structure of the SOP segments 802. Alternatively, a structured format (e.g., JSON) structure matching the SOP segments 802 structure can be output by the alignment algorithm 818. The structured format structure may partition the data therein into indexed sections that correspond to particular sections of the SOP segments 802.

FIG. 9 shows a specific example of implementing the method 800, according to an embodiment of the invention.

In this example, it is desired to split a long natural language text transcript 902 of an interview of a clinical assessment into a plurality of data segments. The interview has been performed according to an SOP 904. Each segment must fit into the context length of a machine learning model that is to be used to analyse the transcript. Each segment must also include the full excerpt from the transcript that corresponds to a question or section of the SOP 904.

The transcript 902 is split into strided transcript chunks 906 using a window size of 200 words and stride size of 100 to split the transcript into T overlapping chunks, as described previously. A language model is then used to extract a contextual embedding for each transcript trunk to produce data chunk embeddings 908.

The SOP 904 is split into its S constituent questions and the interviewer's script for each question is extracted to form a plurality scripts 910 corresponding to individual questions in the SOP 904. A language model is then used to extract a contextual embedding for each SOP script 910 to produce a plurality of script embeddings 912.

These steps are indicated by the top-left panel of FIG. 9 and could be performed in parallel.

Subsequently, the cosine similarity between each of possible pair of data chunk embedding 908 and script embedding 912 is then calculated to produce numerical pairwise scores 914. That is, each data chunk embedding 910 is compared to each script embedding 912. A matrix of pairwise similarities is produced, of dimensionality (S, T). The pairwise scores in the matrix are illustrated graphically by the top-right panel of FIG. 9, where the rightward arrow indicates pairs of increasing SOP question index and the downward arrow indicates pairs of increasing data strided transcript chunk index in the matrix. Each cell in the diagram illustrates the similarity of a given pair, with darker shades indicating higher similarity.

The next step is illustrated by the bottom-right panel of FIG. 9. A “path” from the (0, 0) (top-left cell) to (S−1, T−1) (bottom-right cell) that maximises the sum of cosine similarities, subject to only allowing down-moves (next chunk) and right-moves (next segment), is determined. This guarantees that each segment is matched to one contiguous chunk span, that segment ordering strictly matches the SOP ordering, and that adjacent segments overlap by one chunk. This is one way of providing an alignment algorithm 818 as described with respect to the method 800.

Determining this path can be performed by dynamic programming rather than a brute force search. An example of pseudo-code for an algorithm to determine the path in this way is below shown.

```
function findMaxPath(matrix):
  S = length(matrix)
  T = length(matrix[0])
  dp = empty 2D array of size (S, T)
  path = empty 2D array of size (S, T)
  dp[0][0] = matrix[0][0]
  path[0][0] = [(0, 0)]
  for i from 1 to S−1:
    dp[i][0] = dp[i−1][0] + matrix[i][0]
    path[i][0] = path[i−1][0] + [(i, 0)]
  for j from 1 to T−1:
    dp[0][j] = dp[0][j−1] + matrix[0][j]
    path[0][j] = path[0][j−1] + [(0, j)]
  for i from 1 to S−1:
    for j from 1 to T−1:
      if dp[i−1][j] > dp[i][j−1]:
        dp[i][j] = dp[i−1][j] + matrix[i][j]
        path[i][j] = path[i−1][j] + [(i, j)]
      else:
        dp[i][j] = dp[i][j−1] + matrix[i][j]
        path[i][j] = path[i][j−1] + [(i, j)]
  return path[S−1][T−1]
```

Once an optimal path has been determined, as indicated by the bottom right panel in FIG. 9, a plurality of segments of the transcript can be created.

Since only the interviewer script and not the patient response (which comes after the script) are matched, to create the final segments the chunk matches for segment i are augmented with all the chunk matches for segment i+1 for i<S−1. The output is: for each SOP segment, the transcript excerpt starting from the start of the first matched transcript chunk up to the end of the last matched transcript chunk.

The above-described methods 200-900 have assumed that the machine learning models and the participants in the clinical interview are all operating in the same spoken language. However, machine learning models are generally in a specific language, commonly English. Therefore, it would be desirable to perform the above tasks for clinical assessments performed in languages other than in English.

In a similar manner to the method 200 for performing transcription, an SOP can be provided as an input that conditions a machine learning model configured to translate a text or audio input in one language to an equivalent text and/or audio input in another language more effectively in the context of the clinical assessment. This can be used as a pre-processing step before performing the methods for rating, reviewing, and/or administering clinical assessments described above. For example, input assessment data can be provided to a translation machine learning model together with an SOP to convert a transcription produced by a machine learning model designed for Spanish into an English transcription. The SOP conditions the translation model to provide an English translation that is weighted more heavily towards terms relevant to the clinical assessment of the SOP. This English translation can subsequently be used in the methods above for segmentation, diarisation, rating, review and administration. It would be appreciated this enables these aspects of clinical assessments to be performed automatically in any language.

In other examples, the higher-quality translation produced by SOP conditioning may be provided to a human clinician for rating or review.

In the method 700, the pre-processing step of translation can be performed in each iterative streaming cycle to interact with non-English speakers (for example) even if the generative ML model 708 is designed for or performs optimally in English.

Some machine learning models may be able to transcribe and translate simultaneously. Conditioning such a model on English template data, while giving it a non-English input, allows the model to transcribe and translate at the same time in a way where the translation is more fit for rating by the English rating system than a typical translation would be.

1. A computer-implemented method for performing a clinical assessment, the computer-implemented method comprising:
providing a first input to a machine learning model, the first input comprising template data encoding a template for carrying out a part of the clinical assessment;
providing a second input to the machine learning model, the second input comprising assessment data recorded during the clinical assessment;
wherein the first input is provided to the machine learning model to condition the machine learning model to provide an output based on the second input for use in the clinical assessment; and
using the output from the machine learning model to perform the clinical assessment.

2. The computer-implemented method of claim 1, wherein the assessment data encodes a response of a subject during the clinical assessment, wherein the computer-implemented method comprises using the output to monitor or diagnose a health condition of the subject.

3. (canceled)

4. The computer-implemented method of claim 1, wherein the machine learning model comprises a generative machine learning model.

5. (canceled)

6. (canceled)

7. (canceled)

8. The computer-implemented method of claim 1, wherein the clinical assessment comprises a task for assessing a cognitive function or a neurological health condition of a subject and the assessment data encodes a response of the subject during the task.

9. (canceled)

10. The computer-implemented method of claim 1, wherein the assessment data comprises one or more of audio data, text data, video data, image data.

11. The computer-implemented method of claim 1, wherein the template for administering the clinical assessment comprises one or more of:
instructions for performing the clinical assessment;
an output schema indicating how the output of the machine learning model should be formatted; and
example responses provided by a subject or administrator to tasks within the clinical assessment.

12. (canceled)

13. The computer-implemented method of claim 1, wherein the first input and second input are combined and input into the machine learning model, such that the model is conditioned on content of the template to provide an adapted output in which probabilities of possible outputs are adjusted in view of the template.

14. (canceled)

15. (canceled)

16. (canceled)

17. (canceled)

18. The computer-implemented method of claim 1, wherein the clinical assessment comprises a speech-based clinical assessment comprising tasks instructed by a human or computer-implemented administrator and spoken responses to the tasks provided by a subject; wherein:
the template of the first input comprises text data defining intended content of the clinical assessment;
the assessment data of the second input comprises speech data encoding a response of the subject to an instructed task, where speech data comprises one or both of text and audio data;
the machine learning model is a generative machine learning model trained to generate, based on the second input, an output usable to monitor or diagnose a health condition of the subject; and
the computer-implemented method comprises conditioning the machine learning model on the first input to bias the machine learning model to adapt the generated output in view of knowledge of the intended content of the clinical assessment.

19. The computer-implemented method of claim 18, wherein the output comprises a transformed version of the assessment data usable to monitor or diagnose a health condition, preferably wherein the output comprises one or more of:
a transcription of the speech data;
a diarised version of the speech data, where sections of the speech data are attributed to different participants in the clinical assessment data; and
a segmented version of the speech data, in which the speech data is segmented according to a structure of the clinical assessment defined in the template.

20. (canceled)

21. The computer-implemented method of claim 18, wherein the assessment data comprises audio data encoding speech recorded during the clinical assessment and the machine learning model comprises a transcription model, the transcription model comprising a generative audio-to-text model trained to output text data comprising a transcript of the speech, the method comprising:
conditioning the transcription model by inputting template data encoding one or both of a script for the speech-based assessment and a sample subject response, thereby conditioning the model to assign a higher probability to words more likely to be produced during the task, preferably wherein the template data includes a sample patient response including disfluencies to condition the transcription model to include disfluencies in the transcription.

22. (canceled)

23. The computer-implemented method of claim 1, wherein the machine learning model comprises a rating model, the rating model comprising a machine learning model for outputting a rating indicating performance of a subject in an assessment task based on the assessment data, the method comprising:
providing a first input to the rating model, the first input comprising template data encoding one or both of an administration template comprising an intended format of the clinical assessment and a rating template comprising instructions for rating a subject's response to an assessment task;
providing a second input to the machine learning model, the second input comprising assessment data encoding the subject's response to an assessment task;
wherein the first input is provided to the machine learning model to condition the rating model to provide a rating based on the assessment data in view of the template data; [and] receiving a rating of an assessment task; and
outputting an indication of a health condition of the subject based on the rating of the assessment task.

24. (canceled)

25. The computer-implemented method of claim 1, wherein the machine learning model comprises an administration model, the administration model comprising a machine learning model for automating the instruction of one or more tasks for monitoring or diagnosing a health condition of a subject, the method comprising:
providing a first input to the administration model, the first input comprising template data encoding an administration template comprising instructions for administering a part of the clinical assessment; and
providing a second input to the machine learning model, the second input comprising assessment data recorded during the clinical assessment, the assessment data comprising data encoding a response of the subject to a task administered by the machine learning model;
wherein the administration model maps the second input to a structured output usable to initiate an action to administer the clinical assessment; and
wherein the administration model is conditioned on the first input so that its outputs are determined in view of the administration template.

26. (canceled)

27. The computer-implemented method of claim 25, wherein the administration model is trained to generate a structured text output encoding the action to call, the structured text output preferably comprising a structured JSON format.

28. The computer-implemented method of claim 27, further comprising:
inputting the output of the administration model into a speech synthesis model, the speech synthesis model comprising a text-to-audio generative machine learning model trained to output synthesised speech based on a text input;
such that the administration model outputs text encoding instructions to the subject based on the received response of the subject encoded in the assessment data, and the speech synthesis model generates an audio stream comprising instructions to the subject, thereby facilitating automated audio-verbal administration of the clinical assessment.

29. The computer-implemented method of claim 28, further comprising:
receiving a real-time stream of assessment data during the clinical assessment; and
inputting sequential sections of the assessment data into the administration model in order to generate actions to administer the clinical assessment in real-time.

30. The computer-implemented method of claim 29, wherein the stream of assessment data comprises audio data, the computer-implemented method further comprising:
inputting sequential sections of the audio data into a transcription model, the transcription model comprising a generative machine learning model trained to output text data comprising a transcript of an input section of audio data, wherein the transcription model is conditioned on the first input; and
inputting the text data output by the transcription model into the administration model, wherein the administration model is a text-to-text generative model trained to output structured text for initiating an action to administer the clinical assessment.

31. (canceled)

32. (canceled)

33. (canceled)

34. (canceled)

35. The computer-implemented method of claim 1, wherein the second input comprises one or both of: a video recording of the clinical assessment and image data related to a drawing-based task of the clinical assessment.

36. The computer-implemented method of claim 1, further comprising providing a third input to the machine learning model, the third input comprising a first rating indicating a subject's performance in an assessment task, the first rating suitable for monitoring or diagnosing a health condition;
wherein the template data includes instructions for reviewing the first rating, the second input includes assessment data including a subject response to a task of the clinical assessment, and the output comprises a review rating that evaluates the quality of the first rating,
wherein the assessment data further comprises a rating sheet completed by an administrator and used in providing the first rating, where the template data comprises instructions for checking the rating sheet.

37. (canceled)

38. The computer-implemented method of claim 1, comprising:
encoding each segment of the template data into a respective representation;
splitting the assessment data into a plurality of sections and encoding each section into a respective representation;
using a pairwise scoring algorithm to compute a similarity of each of the template segment representations with each of the assessment data representations;
using an alignment algorithm to determine an optimal alignment of the plurality of sections of the assessment data with the segments of the template using the computed similarity between the template segment representations and the assessment data representations;
using the optimal alignment to split the assessment data into segments corresponding to segments of the template; and
providing one of the segments of the assessment data as an input to the machine learning model for analysing the assessment data.

39. A system for analysing a clinical assessment, the system comprising a processor configured to perform steps comprising:
providing a first input to a machine learning model, the first input comprising template data encoding a template for carrying out a part of the clinical assessment;
providing a second input to the machine learning model, the second input comprising assessment data recorded during the clinical assessment;
wherein the first input is provided to the machine learning model to condition the machine learning model to provide an output based on the second input for use in the clinical assessment; and
using the output from the machine learning model to perform the clinical assessment.

40. (canceled)

41. (canceled)
